(19) United States
(12) Patent Application Publication
(10) Pub. No.: US 2026/0142040 A1
(43) Pub. Date: May 21, 2026

(54) Accurate, computationally tractable, and patient-specific blood flow modeling to improve nanoparticle-based targeted drug delivery

(71) Applicant: The Board of Trustees of the University of Illinois, Urbana, IL (US)

(72) Inventors: Arif Masud, Champaign, IL (US); Shoaib A. Goraya, Urbana, IL (US); Mariam K. Arif, Urbana, IL (US)

(21) Appl. No.: 19/393,324

(22) Filed: Nov. 18, 2025

Related U.S. Application Data
(60) Provisional application No. 63/721,871, filed on Nov. 18, 2024.

(51) Int. Cl. G16H 50/50; G16H 20/10

(52) U.S. Cl. CPC G16H 50/50; G16H 20/10

(57) ABSTRACT

Systems and methods are provided to accurately model nanoparticle deposition in human or animal vasculature while reducing computational costs though a number of specific algorithmic improvements. These methods can be used to virtually evaluate the effects of variations in the nanoparticle configuration (e.g., size, geometry, binding target and/or affinity) and/or timing of delivery (e.g., relative to a patient's circadian rhythm), allowing those properties to be improved in order to increase the nanoparticle dose delivered to an anatomical target and/or to reduce side effects by reducing the total nanoparticle dose used to obtain a specified therapeutically effective level of deposition at the anatomical target. The accuracy and reduced computational cost of these methods allows the simulation to be re-run based on individual patients' data (e.g., MRI or otherwise-obtained maps of vascular geometry), allowing the nanoparticle configuration and/or dosing to be adapted on a per-patient basis.

### CROSS-REFERENCE TO RELATED APPLICATION

The present application claims priority to U.S. Provisional Patent Application No. 63/721,871 filed Nov. 18, 2024, the entire contents of which are incorporated by reference into the present application.

### STATEMENT REGARDING FEDERALLY SPONSORED RESEARCH

This invention was made with government support under 1R01GM135921-01 awarded by the National Institutes of Health. The government has certain rights in the invention.

### BACKGROUND

Nanoparticles show great promise for highly targeted and tailored drug delivery. In contrast with small molecule, biologic, or other relatively simpler therapeutics, nanoparticles contain pharmaceutically active compounds in a micelle or other nano-scale containing structure. Ligands or other substances can be coupled to this structure to improve the anatomical specificity of the nanoparticles (e.g., to facilitate their binding to surface receptors characteristic of target tissues, to prevent drug release except in the presence of receptors or other substances characteristic of target tissues), to control a dosing profile of the nanoparticles, to facilitate externally-controlled drug release or secretion of the particles, or to provide some other benefit. However, the size of such particles introduces complexity into their dosing and delivery, as they do not ‘dissolve’ into the blood as small molecules or biologics do, and furthermore their delivery (and washing) characteristics are more sensitive to the complex micro-scale hydrodynamic processes within the vasculature. While it is possible to model these processes (for specific patients, or for representative ‘mean’ patients) to inform nanoparticle design, simulations of sufficient accuracy to do so are computationally expensive, or even fundamentally intractable, to develop and execute.

### SUMMARY

In a first aspect, a method is provided that includes: (i) determining a spatial pattern of deposition of nanoparticles in a branched vascular structure by simulating a flow of blood bearing the nanoparticles through the branched vascular structure, wherein simulating the flow of the blood comprises simulating nanoparticle flux between the flow of blood and an interior surface of the branched vascular structure as a flux of the nanoparticles (a) between a bulk volume of the flow of blood and a boundary volume proximate to the interior surface and (b) between the boundary volume and the interior surface; and (ii) based on the spatial pattern of deposition of the nanoparticles, determining a property of the nanoparticles or a timing of providing the nanoparticles to a patient to increase an amount of the nanoparticles that are distributed to an anatomical target within the branched vascular structure.

In another aspect, a non-transitory computer readable medium is provided having stored thereon program instructions executable by at least one processor to cause the at least one processor to perform any of the above methods.

In another aspect a system is provided that includes: (i) at least one processor; and (ii) a non-transitory computer-readable medium, having stored therein instructions executable by the at least one processor to cause the system to perform any of the above methods.

These as well as other aspects, advantages, and alternatives will become apparent to those of ordinary skill in the art by reading the following detailed description with reference where appropriate to the accompanying drawings. Further, it should be understood that the description provided in this summary section and elsewhere in this document is intended to illustrate the claimed subject matter by way of example and not by way of limitation

### BRIEF DESCRIPTION OF THE FIGURES

The patent or application file contains at least one drawing executed in color. Copies of this patent or patent application publication with color drawings will be provided by the Office upon request and payment of the necessary fee.

FIG. 1 depicts experimental results, according to an example embodiment.

FIG. 2 depicts aspects of an experimental apparatus, according to an example embodiment.

FIG. 3 depicts experimental results, according to an example embodiment.

FIG. 4 depicts experimental results, according to an example embodiment.

FIG. 5 depicts experimental results, according to an example embodiment.

FIG. 6 depicts aspects of an experimental simulation, according to an example embodiment.

FIG. 7 depicts experimental results, according to an example embodiment.

FIG. 8 depicts experimental results, according to an example embodiment.

FIG. 9 depicts experimental results, according to an example embodiment.

FIG. 10 depicts aspects of an experimental simulation, according to an example embodiment.

FIG. 11 depicts experimental results, according to an example embodiment.

FIG. 12 depicts experimental results, according to an example embodiment.

FIG. 13 depicts aspects of an experimental simulation, according to an example embodiment.

FIG. 14 depicts experimental results, according to an example embodiment.

FIG. 15 depicts experimental results, according to an example embodiment.

FIG. 16 depicts aspects of an experimental simulation, according to an example embodiment.

FIG. 17 depicts experimental results, according to an example embodiment.

FIG. 18 depicts experimental results, according to an example embodiment.

FIG. 19 depicts experimental results, according to an example embodiment.

FIG. 20 depicts experimental results, according to an example embodiment.

FIG. 21 depicts experimental results, according to an example embodiment.

FIG. 22 depicts experimental results, according to an example embodiment.

FIG. 23 illustrates a flowchart of an example method.

### DETAILED DESCRIPTION

The following detailed description describes various features and functions of the disclosed embodiments with reference to the accompanying figures. The illustrative embodiments described herein are not meant to be limiting. It may be readily understood that certain aspects of the disclosed embodiments can be arranged and combined in a wide variety of different configurations, all of which are contemplated herein.

To that end, example methods, devices, and systems are described herein. It should be understood that the words “example” and “exemplary” are used herein to mean “serving as an example, instance, or illustration.” Any embodiment or feature described herein as being an “example” or “exemplary” is not necessarily to be construed as preferred or advantageous over other embodiments or features unless stated as such. Thus, other embodiments can be utilized and other changes can be made without departing from the scope of the subject matter presented herein.

Accordingly, the example embodiments described herein are not meant to be limiting. It will be readily understood that the aspects of the present disclosure, as generally described herein, and illustrated in the figures, can be arranged, substituted, combined, separated, and designed in a wide variety of different configurations. For example, the separation of features into “client” and “server” components may occur in a number of ways.

Further, unless context suggests otherwise, the features illustrated in each of the figures may be used in combination with one another. Thus, the figures should be generally viewed as component aspects of one or more overall embodiments, with the understanding that not all illustrated features are necessary for each embodiment.

Additionally, any enumeration of elements, blocks, or steps in this specification or the claims is for purposes of clarity. Thus, such enumeration should not be interpreted to require or imply that these elements, blocks, or steps adhere to a particular arrangement or are carried out in a particular order.

Unless clearly indicated otherwise herein, the term “or” is to be interpreted as the inclusive disjunction. For example, the phrase “A, B, or C” is true if any one or more of the arguments A, B, C are true, and is only false if all of A, B, and C are false

### I. OVERVIEW

Nanoparticles show significant promise as a delivery vehicle for pharmaceuticals and other therapeutics. They can allow for improved target specificity (both with respect to particle deposition and target-specific payload release), controlled dosing profiles (including sensitivity to later-dosed control molecules and/or specific triggers from target tissues), controlled excretion, or other properties. These improvements may be related to the increased size and complexity of the nanoparticles relative to, e.g., small molecules or biologics, allowing multiple ligands, enzymes, or other elements to incorporated thereon/therein to facilitate various functions.

However, the increased size of such nanoparticles also increases the complexity of their interaction with the endovasculature and blood. Rather than being dissolved in the blood or exhibiting behavior that can accurately be approximated by dissolution (e.g., as small molecules can), the size and geometry of nanoparticles exhibit complex interactions with bulk flow, shear, viscosity, and other properties of surrounding blood. These interactions make it more difficult to accurately estimate the rate of deposition of nanoparticles at any specific location with a vascular structure, and also implicate the modeling of nanoparticle washing/release in order to accurately estimate the time-varying profile of surface-bound nanoparticles across the vascular structure. To accurately estimate such behaviors, it is necessary to accurately represent the full fluid dynamics behavior of the vascular structure and the blood and nanoparticles therein as well as to accurately represent the binding/unbinding of the nanoparticles to the vascular wall in a manner that reflects the complex effects of fluid dynamics and surface geometry (e.g., roughness), which both strongly affect binding/unbinding. These processes span vastly different spatial and temporal scales, leading to naïve approaches that are computationally expensive, or even simply intractable using contemporary computational resources.

The embodiments described herein include specific methods to reduce to computational cost of obtaining physiologically-relevant and highly accurate estimates of the pattern (over time and space) of deposition/release of nanoparticles. These embodiments include implementing specific simplifying assumptions in specific ways in order to significantly reduce computational cost while still obtaining a highly accurate representation of the true behavior of the specific biological system of interest (e.g., the specific branched vascular structure, exposed to blood flow at the specific observed pressure/flow profile, of a specific patient when dosed with a specific nanoparticle). The embodiments described herein reduce the computational cost of performing accurate simulations to a sufficient degree that can be re-run many times, varying properties of the nanoparticles and/or their dosing to improve, for a specific patient or for a class of patients. The results of these variations can then be used to select a specific nanoparticle configuration/dose schedule.

This can be done to increase the deposition of the nanoparticles at one or more anatomical targets (e.g., tumors, specific normal tissues, the base(s) of stenosed or blocked arteries), to reduce deposition at non-desired sites, to reduce side effects (e.g., by targeting the deposition at a target site, thereby allowing the total dose needed to obtain a therapeutic effect to be reduced and thus reducing side effects), and/or to obtain some other benefit. The reduced computational cost also facilitates re-performing such simulations for specific patients (e.g., based on MRI or other accurate scans of a specific patient's specific vascular anatomy, based on a specific patient's observed pattern of blood flow and pressure or other measurements related to the specific patient's circadian rhythm), improving outcomes.

Adapting the timing of dosing can include selecting a time to provide one or more doses of the nanoparticles in order to, e.g., increase deposition at one or more target sites within the patient's vasculature. For example, overall blood flow rate and pressure decrease during sleep, so dosing a set of nanoparticles at a specified time before sleep could increase an amount of the nanoparticles that are deposited at a target site (and/or increase an amount of time that nanoparticles remain at the target site after deposition and prior to being dislodged by blood flow) in a manner that is related to, e.g., reduced turbulence, flow shear, or other properties of the blood at the target site during sleep. Determining a timing of dosing could include determining a dose timing relative to a circadian rhythm of a patient, such that administering the dose could include measuring the phase of the patient's circadian rhythm (e.g., based on sleep/wake times, patterns of blood pressure over the day, etc.) and then administering the nanoparticles at a timing determined based on the measured circadian rhythm phase. The timing could be determined for a specific patient or could be determined for an ‘average’ patient (e.g., by simulating nanoparticle deposition in a model of an ‘average’ vascular structure), in which case the circadian-relative timing could be determined for the ‘average’ patient but administered based on the measured circadian rhythm phase of a specific patient.

The ‘anatomical target’ or ‘target tissue’ of an embodiment described herein could be any type of tissue, specified relative to a branched vascular structure to be simulated. For example, the target could represent a tumor, electrically abnormal heart muscle, or some other normal or diseased tissue that is sought to be targeted by nanoparticles, e.g., to delivery chemotherapy thereto in a highly targeted fashion. In another example, the target could include portion(s) of the vasculature itself, e.g., to facilitate targeting of vasculature that is stenosed, blocked, or otherwise affected by a stroke or other adverse event, e.g., to restore blood flow thereto by delivering clot-busting drugs, blood thinners, vasodilators, or other substances. In such examples, the target tissue could be proximal portions of such effected vessels, portions of upstream vessels proximal to the affected vessels' branching therefrom (e.g., in examples wherein the blockage of the vessel is represented by deleting the affected vessels from the branch vascular structure), or other targets of drugs to address such blockages.

A simulation of deposition/release of nanoparticles within a branched vascular structure as described herein could be performed using a single representative branched vascular structure (e.g., an average generated from a set of structures for specific patients, a representative structure determined from a single patient and used for other patients) for many patients or using patient-specific structures (e.g., determined based on an MRI or other anatomical scan). Such a simulation could be performed based on a representative set of blood flow, blood pressure, blood viscosity, or other parameters and/or patterns of change thereof throughout the day (e.g., with a characteristic blood pressure and flow cycle that varies across the day according to a circadian rhythm) and/or based on a specific set of such parameters or patterns measured from a specific patient. Accordingly, nanoparticle configurations (e.g., size, binding ligand) and/or dose timing (e.g., relative to the circadian rhythm) could be determined for a representative patient or class of patients or for a specific patient.

The nanoparticle configuration determined from repeated simulation as described herein could be used to generate a corresponding population of nanoparticles that could then be administered to a patient (e.g., on a single-patient basis, to a class of patients that match the underlying simulations in some manner, and/or to all patients based on the use of a single representative vascular structure or other simulation particulars). Additionally or alternatively, the nanoparticle configuration determined from repeated simulation as described herein could be used to select a nanoparticle (or mix of nanoparticles) from a set of available nanoparticles for administration to a patient. For example, the available set of nanoparticles could vary with respect to nanoparticle size, nanoparticle geometry (e.g., sphericity/eccentricity), nanoparticle target (e.g., different nanoparticles having different receptors, antibodies, or other elements for binding to different targets), binding affinity, drag coefficient, and/or other properties and selecting one of the nanoparticles could include selecting the nanoparticle that is closest to the determined properties and/or performing the simulation for each of the available nanoparticles and selecting the one whose outcome (e.g., amount of nanoparticles deposited proximate to the target(s)) is deemed favorable.

As noted above, to accurately model the deposition (and release) of nanoparticles within a vascular structure in the face of realistic time-varying blood flow and pressure profiles (which, themselves, may vary across the circadian cycle), it may be necessary to account for complex fluid dynamics at multiple spatial and temporal scales as well as to account for interactions between the nanoparticles and the walls of the vasculature at the nano scale. To naively simulate such a system across such disparate temporal and spatial scales could be extremely computationally expensive (e.g., with respect to the processor cycles, time, memory, storage, network interconnect bandwidth, power, or other computational costs) or even computationally intractable using contemporary resources. Instead, the specific methods described herein selectively reduce the computational costs of specific aspects of the simulation, providing significant computational cost saving while retaining accuracy.

The embodiments described herein include performing simulation of complex fluid blood flows within a simulated branched vascular structure (e.g., a structure determined from MRI, angiographic, or other measurements of one or more patient's cerebrovascular, cardiovascular, or other vascular structure(s)), the flows driven by pulsatile flow/pressure at one or more input vessels of the structure to simulate the natural pulsatile drive of blood flow by the heart. These simulations can render the velocity (e.g., as a 3-vector field) and kinematic pressure (e.g., as a scalar field) within the fluid throughout the vascular structure. These simulations can also account for the nanoparticles flowing within the bulk of the blood as a concentration (e.g., as a scalar field). The blood can be simulated as an incompressible fluid that exhibits shear-thinning or otherwise exhibits a shear-dependent viscosity (e.g., as a shear-rate dependent fluid whose stress tensor depends on the fluid viscosity). Such a method exhibits significant computational cost savings relative to, e.g., simulating the movement of individual nanoparticles within the fluid.

Another specific improvement described herein is to simulate only the arterial side of the vascular structure, representing the venous side as simplified hydrodynamic resistances at the end of each terminal branch of the arterial vascular structure. By doing so, the computational cost of the simulation can be approximately halved, since the ‘venous half’ of the simulation need not be calculated. This could include representing the resistance at the end of each terminal vessel of the arterial vascular structure as a constant fluid resistance, e.g., as a mean pressure at the terminal vessels divided by the mean flow rate therethrough, with the mean taken across a generic cardiac flow cycle. Addition boundary conditions at the terminal vessels (e.g., pressure exerted from the non-simulated venous vascular structure) could be determined from this simplification.

Another specific improvement described herein is to simulate the movement of nanoparticles into and out of the boundary layer of blood (i.e., a thin layer of the blood proximate to the vascular wall) as a single compartment rather than as the movement of individual nanoparticles interacting with the wall and with the fluid flow (e.g., drag, shear) in the boundary layer. The movement of nanoparticles between the bulk of the blood volume and the boundary layer and between the boundary layer and the vessel walls (e.g., as binding events and, potentially later, as un-binding events as the blood flow washes the bound nanoparticles away) can thus be accurately accounted for without the significant computational costs of directly simulating such interactions. Such a naïve direct simulation would require substantial computational resources since such mechanisms operate from large scales (e.g., millimeters) down to several orders of different small scales (e.g., nanometers), implicating significantly more nodes of a finite-element simulation or similarly increased costs if implemented in some other manner.

The theoretically and experimentally validated simulations of the boundary layer described herein may be implemented in a variety of ways (e.g., as a Robin boundary condition at the vessel walls) to accurately simulate the behavior of nanoparticle deposition/release. This could include using a Robin boundary condition or other boundary-layer simulation that simulates the rate of adhesion (or dis-adhesion) of nanoparticles to the vessel wall as concentration-dependent function of a deposition parameter that is, itself, a parameterized function of the fluid shear and/or shear rate in the fluid proximate to the vessel wall (e.g., in the boundary layer). This function may be experimentally validated such that parameters like nanoparticle size or geometry can be easily varied. Additionally, the parameterized function could be specified to account for experimentally-evaluated vessel wall surface roughness (e.g., asperity), which can affect nanoparticle adhesion/release.

By simplifying the boundary between blood and vessel wall in this way, the dynamics of nanoparticle transport therebetween (i.e., between the concentration of nanoparticles in the blood proximate to a specific area of vessel wall and the concentration of nanoparticles adhered to that specific area of vessel wall) can be accurately simulated in a manner that depends on a limited number of properties of the simulated blood flow proximate to the area of vessel wall (e.g., to shear or shear rate and nanoparticle concentration or concentration gradient), thereby significantly reducing the computational cost (e.g., relative to a more direct per-nanoparticle simulation, which would require segmenting the vascular geometry at a much smaller spatial scale). Additionally, the parameterization of the equations governing nanoparticle transport at the boundary could include parameters for particle size, geometry, target affinity, drag, or other nanoparticle properties, roughness or other properties of the vessel wall, or other properties of the vascular system of interest, facilitating the variation of such parameters in order to determine improved nanoparticle configurations for targeted delivery thereof.

### II. EXAMPLE EMBODIMENTS

Provided herein are example model(s) and aspects thereof that can provide accurate simulation of nanoparticle deposition/release within anatomically detailed representations of human or animal vasculature, blood flow characteristics, and/or circadian rhythm (e.g., of a specific patient, or of a representative patient). These models can, in a computationally tractable manner, provide accurate simulations of the pattern (spatially and over time) of the deposition/release of nanoparticles within the representation, allowing variations in nanoparticle configuration (e.g., size, geometry, binding characteristics) and nanoparticle dosing (e.g., dose size, dose timing relative to patient circadian rhythm) to be simulated repeatedly. These repeated simulations can allow the properties of the nanoparticle and/or of their dosing to be improved (e.g., optimized) for a specific patient and/or population of patients. This can facilitate increasing the therapeutic effect (e.g., by increasing nanoparticle deposition and retention in target tissues) and/or reducing side effects (e.g., by reducing nanoparticle deposition and/or retention in non-target tissues and/or the blood while achieving a therapeutically sufficient deposition and/or retention of nanoparticles in the target tissue).

### Example Embodiments and Experimental Results

Embodiments described herein can include delivery of drugs or other substances using nanocarriers tethered with vasculature-targeting epitopes. This can be done to increase the therapeutic efficacy of the drug while reducing the drug side effects, e.g., by reducing the amount of drug needed to obtain a desired therapeutic effect. The circadian rhythm, which is governed by the central nervous system, can be leveraged for targeted drug delivery due to sleep-wake cycle changes in blood flow dynamics. Embodiments described herein include the use of an advanced fluid dynamics modeling method to more accurately model drug transport (and thus to deliver drugs at more efficacious point(s) in time) that is based on viscous incompressible shear-rate fluid (blood) coupled with an advection-diffusion equation to simulate the formation of drug concentration gradients in the blood stream and buildup of payload at the targeted site(s). These methods can be experimentally calibrated to a nanoparticle-endothelial cell adhesion model that employs Robin boundary conditions to describe nanoparticle retention based on probability of adhesion, a friction model that accounts for surface roughness of endothelial cell layer, and a dispersion model based on Taylor-Aris expression for effective diffusion in the boundary layer. This methods was experimentally validated and then tested on engineered bifurcating arterial systems where impedance boundary conditions were applied at the outflow to account for the downstream resistance at each outlet. These methods were also applied to a virtual geometric model of an in vivo arterial tree developed through MRI-based image processing techniques. These experiments verified the utility of the embodiments described herein with respect to modeling drug transport, adhesion, and retention at multiple sites in virtual in vivo models. These embodiments provide a platform for simulating circadian rhythm modulated blood flow for targeted drug delivery, allowing drugs delivery schedules and/or properties of the drug delivery vector itself to be tailored to specific patients while reducing in vivo experimentation.

Diseases of the central nervous system (CNS) are among the leading causes of death in the United States, with Alzheimer's disease alone being responsible for 121,499 deaths in 2019. Even in non-fatal cases, these CNS conditions pose a tremendous health burden. Treatment of these diseases is limited in part by the difficulty of delivering therapeutics to the site of the diseased tissue due to the blood-brain barrier (BBB). It Is desirable to improve the delivery of therapeutic drugs of interest to diseased tissues through the bloodstream while reducing unwanted interaction with healthy tissues and subsequent adverse reactions. Drug molecules can be encapsulated within biodegradable nanoparticles tethered with bioactive ligands that are capable of binding to receptors that are overexpressed in the microvascular networks of target tissues. The mechanism of the transport across the BBB appears to be receptor-mediated endocytosis followed by transcytosis of such drug-loaded nanoparticles into the brain or by the release of the drugs within the endothelial cells. Nanoparticle surface treatment can be employed to facilitate such receptor-mediated uptake. The pharmacokinetics and/or the efficacy of drugs can be enhanced by timing drug delivery to the patient's circadian rhythm and/or to the 24-hour cycle of the day.

The circadian rhythm governs the 24-hour cycle of bodily changes impacting sleep, metabolism, and blood flow in response to natural cues. Disruptions to this cycle correlate with adverse health outcomes like increased risk of myocardial infarction/stroke and worsened eczema severity due to sleep disruptions. Circadian rhythms also affect drug metabolism enzymes, with implications for targeted drug delivery, especially in chronotherapy. Chronotherapy involves administering drugs at specific times to optimize effectiveness and minimize side effects based on circadian cycles and physiological changes. Circadian rhythms influence the pharmacokinetics and pharmacodynamics of medications, suggesting potential improvements in therapeutic impact by aligning drug delivery with circadian regulation. Synchronizing targeted drug delivery with the circadian clock can enhance efficacy, e.g., through personalized medicine, reducing the risk of adverse effects. Peptide-ligated nanoparticle drug delivery systems, responsive to circadian cues, offer a means to release drugs at optimal times, improving therapeutic impact.

To simulate the intricate interactions between nanoparticles and biological systems, thereby facilitating the tailoring of drug delivery vectors and/or drug delivery schedules to specific patients, a computational model is provided herein of nanoparticle-facilitated drug delivery. Such a model can be used to quantify the factors that affect particle transport and retention at targeted sites under circadian blood flow fluctuations in specific patients. PLGA-b-HA nanoparticles decorated with VHSPNKK peptides or other drug delivery vectors can be represented in the model as a homogenized mixture in the fluid and an advection-diffusion equation can be used to simulate the development of drug concentration gradients in the blood flow. A specific integration of the Taylor-Aris dispersion model and an asperity model with Decuzzi's particle-cell adhesion model can be used to accurately predict nanocarrier transport, attachment, and detachment. Such model(s) can account for shear-induced diffusion of particles in the boundary layer, while the asperity model can account for the interaction between the fluid and endothelial cell layer. The computational methods described herein offers a specific, highly accurate, personalized, and computationally tractable approach by integrating mechanics-based drug delivery models into blood flow modeling approaches in patient-specific geometries.

The drug delivery model can be implemented in a stabilized finite element framework to accurately capture concentration gradients in convection-dominated flow conditions. Blood rheology can be accounted for via a shear-rate-dependent model. Circulatory effects downstream of the modeled arterial system can be incorporated using resistance outflow boundary conditions. Physiologically relevant pulsatile inflow rates and pressure profiles can be used to determine resistance functions. Circadian variation in hemodynamics can be modeled using clinical data, e.g., measured from specific patients. A particle-cell adhesion model can be embedded via Robin boundary conditions (BC) applied at the endothelial cell layer surface. Robin BC combine Neumann (drug flux in normal direction) and Dirichlet (drug concentration) boundary conditions through the vascular deposition parameter. This parameter accounts for particle-cell binding kinetics by integrating information of biochemical, biophysical, and geometric properties from micro/nano scale into macro scale.

The methods described herein were validated experimentally. In vitro experiments were carried out in a 3D microfluidic chamber, lined with TNF-α treated mouse endothelial cells with enhanced VCAM expression, to determine particle adhesion and retention at the target site. Atomic force microscopy (AFM) was used to determine the binding affinity between coated nanoparticles and the ligand-coated receptor cells. This data was used to set the biochemical parameters in the probabilistic particle-cell adhesion model. The validated model was then employed to simulate the effect of circadian modulation of blood flow on targeted drug delivery via nanoparticles in virtual in vivo geometries.

### Governing Equations for Targeted Drug Delivery in Vasculature

Example balance laws for transport of ligand-coated nanoparticles in an incompressible shear-rate dependent fluid (e.g., as blood) are given below:

$${u}_{, t} + u \cdot ∇ u = - ∇ p + ∇ \cdot {σ}_{v} ( u ) + {f}_{b} in Ω \times ] 0 , T [ \qquad ( 1 )$$

$$∇ \cdot u = 0 in Ω \times ] 0 , T [ \qquad ( 2 )$$

$${C}_{, t} + u \cdot ∇ C - ∇ \cdot ( κ \cdot ∇ C ) = f in Ω \times ] 0 , T [ \qquad ( 3 )$$

asd where u(x,t) and p(x,t) are the velocity and kinematic pressure fields, respectively; C (x,t) is the volume concentration of particles; Υν is the deviatoric stress tensor which in the case of shear-rate dependent fluids depends on the viscosity of the fluid, and κ is the diffusivity tensor; f_{b} is the body force for the mechanical field, and f is the source/sink term for the concentration field. Equation (1) is the momentum balance equation, Equation (2) is the continuity equation for conservation of mass which enforces the incompressibility constraint, and Equation (3) is the convection-diffusion equation for the evolution of the particle concentration field.

Such a mathematical model can be supplemented with initial conditions in the domain x∈Ω at time t=t0 and boundary conditions on the domain boundary Γ,

$$u ( x , 0 ) = {u}_{0} ( x ) in Ω \times \{ 0 \} \qquad ( 4 )$$

$$C ( x , 0 ) = {C}_{0} ( x ) in Ω \times \{ 0 \} \qquad ( 5 )$$

$$u ( x , t ) = {g}_{M} on {Γ}_{g}^{M} \times ] 0 , T [ \qquad ( 6 )$$

$$C ( x , t ) = \{ \begin{matrix}{g}_{E} for 0 \leq t \leq {t}^{*} \\ 0 for t > {t}^{*}\end{matrix} on {Γ}_{g}^{E} \times ] 0 , T [ \qquad ( 7 )$$

$$σ \cdot n = ( {σ}_{ν} ( u ) - pI ) \cdot n = {h}_{M} on {Γ}_{h}^{M} \times ] 0 , T [ \qquad ( 8 )$$

$$( κ \cdot ∇ C ) \cdot n = {h}_{E} on {Γ}_{h 1}^{E} \times ] 0 , T [ \qquad ( 9 )$$

$$( κ \cdot ∇ C ) \cdot n + \prod C = 0 on {Γ}_{h 2}^{E} \times ] 0 , T [ \qquad ( 10 )$$

where u₀ and C₀ are the initial conditions for velocity and concentration fields, respectively; g_{M} and g_{E} are the prescribed values for velocity and concentration fields, respectively; while h_{M} and h_{E} are the prescribed fluxes for the velocity and concentration fields, respectively. t* denotes the time duration for the injection of particles at the inlet. σ is the total stress in the fluid and n is the outward normal vector at the boundary. The boundaries satisfy the following conditions: Γ^{M}_{g}∩Γ^{M}ₕ=0, Γ^{M}_{g}∪Γ^{M}ₕ=Γ, Γ^{E}_{g}∪Γ^{E}ₕ=0 and Γ^{E}_{g}∩Γ^{E}ₕ=Γ. Equation (10) defines Robin boundary conditions that were used for particle adhesion to the endothelial cell layer. Π is the positive-valued adhesion factor that depends on particle-cell adhesion model.

### Shear-Rate Dependent Blood Flow Model

A shear-rate dependent non-Newtonian model for blood can be employed in the simulations described herein. The flow of shear-thinning fluids generally gives rise to pseudo-plastic velocity profiles that are characterized by lower velocities due to low convective flow but sharper and thinner boundary layers. The ability to capture the boundary layer is of importance for shear-thinning flows.

The Cauchy stress tensor is split into volumetric stress and viscous stress components by treating pressure as an independent field:

$$σ = - pI + {σ}_{ν} ( u ) \qquad ( 11 )$$

where σ is Cauchy stress tensor, p is the pressure field or the volumetric stress, and σᵥ(u) is the nonlinear viscous stress given as:

$${σ}_{ν} ( u ) = 2 μ ( γ ) ϵ ( u ) \qquad ( 12 )$$

where ϵ(u) is the rate-of-deformation tensor which is defined as ϵ(u)=½(∇u+(∇u)^{γ}). In shear rate dependent fluids, the viscosity field μ(γ) is a nonlinear function of the shear-rate γ defined as γ=\sqrt{2ϵ(u):ϵ(u)}.

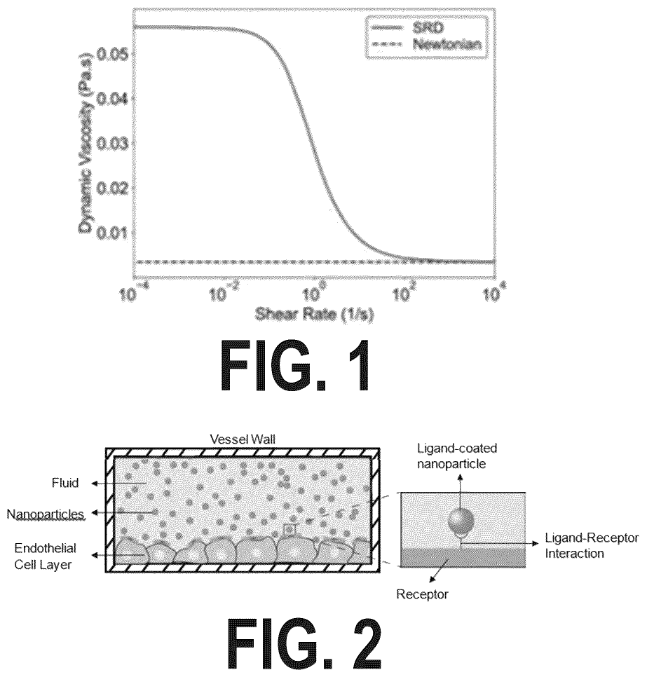

The Carreau-Yasuda model can be used to represent the shear-thinning behavior of blood. In shear-rate dependent fluids, the nonlinear function of the viscosity is given by:

$$μ ( γ ) = μ + {( {μ}_{0} - μ ∞ ) [ 1 + {( λ γ )}^{a} ]}^{( n - 1 ) / a} \qquad ( 13 )$$

where μ₀ and μ∞ are asymptotic viscosities at zero and infinite shear-rate, respectively, and a, n and λ are empirically determined constitutive parameters. Coefficients a and n are non-dimensional parameters that control the shear-thinning or shear-thickening behavior of fluids in the non-Newtonian regime between the two asymptotic viscosities. The Carreau-Yasuda model possesses constant values of viscosity at both low and high ranges of shear-rate, and a varying viscosity in the intermediate range of shear-rate. The model reverts to the Newtonian fluid model by setting μ₀=μ∞ as shown in FIG. 1.

FIG. 1 illustrates the viscosity of Newtonian and shear rate dependent (Carreau-Yasuda) constitutive models for blood. The Carreau-Yasuda model for blood shows changes in viscosity as a function of the shear-rate, while the Newtonian model has a constant viscosity at all shear-rates.

Note that the specific computational models presented herein are general and any constitutive model for shear rate dependent response of the fluid can be additionally or alternatively be used.

### Resistance Boundary Conditions

The cardiovascular system is comprised of heart and blood vessels that form a closed network. Developing a computational model for the entire system is not only difficult due to its geometric complexity, but it is also computationally expensive. Consequently, developing appropriate outflow boundary conditions that can take into account the effects of the circulation system downstream of the zone of interest can provide significant benefits to practical blood flow modeling strategies. With appropriate boundary conditions applied at the outflow surfaces, blood flow simulations can be carried out only in the network of interest, and this can substantially reduce the cost of computation while preserving the accuracy of the modeled physics.

The downstream resistance R can be assumed to have a linear relation between the flow-rate and the pressure. The mean pressures and flow-rates at outlets determine the constant resistance values at these outlets.

$$R = Pmean / Qmean \qquad ( 14 )$$

where Pₘₑₐₙ and Qₘₑₐₙ are the mean pressure and flow-rate for one generic cardiac cycle at the outflow boundary, respectively. The resistance boundary conditions that produce physiological pressure waveform at the outflows are imposed at all the outlets Γₒᵤₜ to incorporate the downstream resistive effects of the blood vessels. It is given as:

$$p = R \text{?} ( u \cdot n ) d Γ + {p}_{0} \qquad ( 15 )$$

$$\text{?} \text{indicates text missing or illegible when filed}$$

where p₀ is the constant downstream pressure. This method also allows for embedding clinically measured downstream resistance via a functional form for the outflow boundary conditions. In this case flow-rate and pressure profiles of a typical carotid artery are used to determine time-dependent resistance functions.

### Particle-Cell Adhesion Model

The ability of the drug coated nanoparticles to adhere to the target endothelial cells is an important aspect of targeted drug delivery processes. According to the continuum modeling approach described above, information at the micro- and nanoscale can be embedded in the computational model via the Robin boundary condition that is applied at the targeted vessel wall Γ_{wall}.

$$( κ \cdot ∇ C ) \cdot n + Π C = 0 at {Γ}_{wall} \times ] 0 , T [ \qquad ( 16 )$$

where Π=P_{aγ}(dₚ/2) is a vascular deposition parameter, γ is the shear rate as defined above, dₚ is the diameter of nanoparticles, and Pₐ is the probability of adhesion. The Robin boundary condition accounts for particle-cell adhesion based on the probability of adhesion Pₐ. This probability can be related to the strength of adhesion: the larger is the value of Pₐ the larger is the adhesive strength of the particle to the endothelial cell layer. Only the particles in the boundary layer of the fluid filled vessel experience the binding affinity (FIG. 1). Thus Equation (16) integrates information from the macroscale (vessel geometry and flow conditions) with data from the micro- and nanoscale (particle geometry, receptor density, and affinity), thereby avoiding massive, computationally inefficient discretization over multiple spatial and temporal scales. Moreover, the mass flux of particles in the direction normal to the wall can be related to the local increase in mass of particles adhering per unit surface φ(x,t):

$${{- ( κ \cdot ∇ C ) \cdot n \vert}_{wall} = - D \frac{\partial C}{\text{?}} \vert}_{wall} = \frac{\partial φ}{\partial \text{?}} ( ∴ \text{?} = DI ) \qquad ( 17 )$$

$$\text{?} \text{indicates text missing or illegible when filed}$$

The probability of adhesion Pₐ represents 2D binding characteristics of receptors and ligand coated particles that are attached to surfaces, and their bonding is subjected to dislodging hydrodynamic forces. An experimentally validated closed form expression can be used for the probability of adhesion. This expression describes cellular adhesion by coupling the bond forces to the binding affinity. The probability of adhesion for ellipsoid particles is given as:

$$\text{?} ≃ \text{?} exp [ - \frac{λ \text{?}}{\text{?} T} ] \qquad ( 18 )$$

$$\text{?} \text{indicates text missing or illegible when filed}$$

where f̅=F_{d/s}/(mᵣA_{c}) is the force per unit ligand-receptor pair, A_{c} is the area of interaction between ligand and receptor, F_{d/s} is the dislodging force due to drag force and rotating torque, Kₐ⁰ is ligand-receptor affinity constant at zero load, mₗ is surface density of ligand molecules (#/m²), and mᵣ is surface density of receptor molecules (#/m²). The dislodging force F_{d/s} also includes the effect of equilibrium separation distance between the cell layer and the particle, and the maximum distance at which ligand-receptor bonds can be formed. The above expression shows that the strength of adhesion is affected by the geometric features of the particle (particle size and aspect ratio), by the biophysical parameters (dislodging force depending on the strength of ligand-receptor bonds and wall shear stress, and the surface densities of the ligands mₗ and of the receptors mᵣ), and by the biochemical parameters (characteristic length λ of the ligand-receptor bond and the characteristic affinity constant Kₐ⁰ of the ligand-receptor pair).

For spherical particles, Equation (18) can be simplified to:

$$\text{?} ≃ \text{?} {π ( {d}_{p} / 2 )}^{2} exp [ - \text{?} \frac{\text{?}}{\text{?}} ] \qquad ( 19 )$$

$$\text{?} \text{indicates text missing or illegible when filed}$$

where the parameters α₁, α₂, and β are given as:

$${α}_{1} = \text{?} ; {α}_{2} = \text{?} [ 1 - ( 1 - \frac{2 Δ}{{d}_{p}} ) ] ; \text{?} = \frac{\text{?} λ {F}^{S}}{{k}_{8} T}$$

$$\text{?} \text{indicates text missing or illegible when filed}$$

In the above parameters, Δ is the distance between particles and endothelial cell layer at equilibrium, λ is ligand-receptor bond length, and F^{S} is a drag coefficient that depends on the particle geometry.

Particle-cell binding is an interrelation of random processes at distinct time scales. Particles undergo rapid binding and transport, resulting in a stochastic spatial distribution of bound particles fluctuating about some mean distribution. It was observed during the experiments that cells were clustered together at random locations giving rise to non-uniform distribution of receptor density. To incorporate this effect into the mathematical framework, a Gaussian sampling of receptor densities can be used over the endothelial cell layer. Gaussian sampling was done from a normal distribution with a coefficient of variation of 0.1. This resulted in a more realistic picture of the particle-cell binding process, which is stochastic in nature. The resulting adhesion model when integrated in the computational framework described herein provides a mechanistic description of the magnitude and distribution of nanoparticles that adhere to the endothelial layer.

The parameters to compute Pₐ in the adhesion model described above were obtained through characterization experiments whose results are reported in Table 1. Atomic force microscopy (AFM) was used to determine the bond-length and the equilibrium distance between the peptide and the protein. Meanwhile, the ligand and receptor densities, as well as the ligand-receptor affinity constant, were adopted from the published literature. The binding affinity was observed to decrease in experiments where particles were not treated with VHSPNKK peptides. This decrease was accounted for in the adhesion model by calibrating the binding affinity constant with the experimental data.

TABLE 1
Parameters for Carreu-Yasuda blood flow model.

| | |
|---|---|
| Parameter | Value |

| 1. | Initial viscosity μ₀ | 0.56 | dyn · s/cm² |
|---|---|---|---|
| 2. | Final viscosity μ_{∞} | 0.0345 | dyn · s/cm² |
| 3. | Model parameter λ | 1.902 | |
| 4. | Model parameter a | 1.25 | |
| 5. | Model parameter n | 0.22 | |
| 6. | Density ρ | 1.055 | g/cm³ |

### Modeling the Surface Roughness of Endothelial Cell Layer

Accurate modeling of particle transport and adhesion can take into account the effect of interaction between fluid in the boundary layer and the irregular surface of the endothelial cell layer. The irregular surface induces roughness that can help in the retention of attached particles. In numerical test cases, an idealized smooth cell surface failed to produce experimentally observed particle retention during detachment/wash flows. Accordingly, a two-body contact formulation was used to model the particle-cell interaction.

The mathematical formulation of the two-body contact problem can be implemented through the use of constraint equations, e.g., the Kuhn-Tucker conditions, to describe the relationship between the contact pressure q_{N} and penetration or gap g_{V} in the surface normal direction. In addition, coupling in the tangential direction can be expressed through conjugate variables, the tangential gap grand the tangential flux q_{T}. The gap functions can be defined as:

$${g}_{N} = - [ X + u ] , \text{?} = - [ \text{?} {n}^{+} - {g}_{N} {n}^{+} ] \qquad ( 20 )$$

$$\text{?} \text{indicates text missing or illegible when filed}$$

where [[⋅]] and [[⋅]] are jump operators along the interface of the two bodies with dot product and tensor product, respectively. The fact that the fluid and cell layer are always in contact with each other can be used to render the relative position vector X=0. Additionally, the cell layer does not move and has zero velocity. This simplifies (20) to:

$$\text{?} = - \text{?} , \text{?} = - \text{?} \qquad ( 21 )$$

$$\text{?} \text{indicates text missing or illegible when filed}$$

where u_{N} and u_{T} are normal and tangential components of the fluid velocity, respectively. Embedding Kuhn-Tucker contact conditions into the convection-diffusion Equation (3) and ignoring the normal component g_{N} yields the following:

$$C {,}_{t} + u \cdot ∇ C - ∇ \cdot ( κ \cdot ∇ C ) + {g}_{T} \cdot {q}_{T} = f \qquad ( 22 )$$

Numerical modeling of asperities (e.g., as shown in FIG. 2) can implicate discretizations that are at least an order of magnitude smaller than the asperity size. Since endothelial cells are in the range of 0.1 μm, it can be appropriate to use a micromechanics asperity model for contact between the fluid and the irregular cell layer surface. We employed the well-known Coulomb friction model at the mean asperity height to model surface roughness due to irregular endothelial cell layer. Coulomb model couples the normal and tangential tractions with a proportionality constant called the friction coefficient μ_{f}. The tangential flux given by the Coulomb model is:

$${q}_{T} = {μ}_{f} ( ∇ C \cdot t ) \qquad ( 23 )$$

where t is a tangential vector at the surface of the cell layer. The friction term in Equation (22) can be limited to activated at a mean asperity height d from the bottom of the vessel (e.g., as depicted in FIG. 2). The friction coefficient between the fluid and the endothelial cell layer can be estimated empirically by calibrating the model Equation (23) with experimental data.

FIG. 2 is a schematic diagram of aspects of drug delivery using nanocarriers designed for active targeting in circulation. For particle-cell adhesion, a Robin boundary condition embedded with an adhesion model can be employed at the cell surface. An asperity model, as well as a Taylor dispersion model, can be operational in the boundary layer to simulate cell surface roughness and dispersion of entrapped particles, respectively.

### Dispersion of Particles in the Boundary Layer

In confined flows, dispersion due to shear (or velocity gradient) can affect the mixing and transport of particles in the boundary layer region (e.g., as depicted in FIG. 2). Dispersion can increase the dislodging hydrodynamic forces, which can in turn impact the number of nanoparticles that are retained at the endothelial cell layer during a washing stage. This effect can be incorporated by using the Taylor-Aris expression for effective diffusion in the boundary layer region, taking into account longitudinal dispersion:

$$\text{?} = D ( 1 + \frac{{P}_{e}^{2}}{48} ) \qquad ( 24 )$$

$$\text{?} \text{indicates text missing or illegible when filed}$$

where Pe=UL/D is the Peclet number, U is the mean flow velocity, and L is the length scale associated with the problem. The length scale chosen is diameter of the particle dₚ. D is isotropic particle diffusivity coefficient obtained from Stokes-Einstein equation D=k_{B}T/(3μdₚ) where k_{B}T is the Boltzmann thermal energy (4.142×10⁻²¹ J) and μ is the dynamic viscosity of the fluid.

### Stabilized Finite Element Method

A variety of different methods (e.g., finite element methods) can be used to implement the mathematical representations provided herein of nanoparticle-bearing blood flowing in branched vascular structures. For example, provided below are details of a stabilized finite element method that provides a fully coupled system of equations that can be consistently linearized to develop a monolithic solution procedure that yields quadratic rate of convergence in nonlinear solution strategies.

### Standard Weak Form

The standard weak form of the problem is: Find V=(u,p,C)∈Ut×Pt×Ct, such that ∀W=(w,q,η)∈

[symbol]

$$( w , u {,}_{t} ) + ( w , u \cdot ∇ u ) + ( ∇ w , {σ}_{v} ( u ) ) - ( ∇ \cdot w , p ) = {( w , {h}_{M} )}_{Γ} Mh + ( w , {f}_{b} ) \qquad ( 25 )$$

$$( q , ∇ \cdot u ) = 0 \qquad ( 26 )$$

$$( η , C {,}_{t} ) + ( η , u \cdot ∇ C ) + ( ∇ η , κ \cdot ∇ C ) = {( η , {h}_{E} )}_{Γ} Eh - ( η , f ) \qquad ( 27 )$$

where

$$\text{?} = \text{?} Ω$$

$$\text{?} \text{indicates text missing or illegible when filed}$$

denotes the L₂(Ω) inner product.

The appropriate functional spaces for the velocity, pressure, and concentration trial solutions, [symbol] are defined below:

$$\text{?} = \{ u ( \text{?} , t ) \vert u ( x , t ) = \text{?} ( x , t ) , x ∈ \text{?} , u ( \text{?} t ) ∈ ( \text{?} ( Ω ) \text{?} \} \qquad ( 28 )$$

$$\text{?} = \{ p ( \text{?} , t \vert p ( x , t ) ∈ ( {L}_{2} ( Ω ) \text{?} \} \qquad ( 29 )$$

$$\text{?} = \{ C ( \text{?} t ) \vert C ( x , t ) = \text{?} ( x , t ) , x ∈ \text{?} C ( \text{?} t ) ∈ ( \text{?} ( Ω ) \text{?} \} \qquad ( 30 )$$

$$\text{?} \text{indicates text missing or illegible when filed}$$

where L₂(Ω) and H¹(Ω) are the standard Sobolev spaces. Let w(x)∈W=(H₀¹(Ω))^{nsd},q(x)∈Q=C⁰(Ω)∩L₂(Ω), and η(x)∈H=H₀¹(Ω) be the weighting functions for the velocity u, kinematic pressure p, and concentration C, respectively. The weighting function spaces W,Q,H are the corresponding spaces of Equations (28)-(30) that satisfy the homogeneous part of the essential boundary conditions.

### Variational Multiscale Method

The bounded domain Ω is discretized into non-overlapping subdomains \tilde{Ω} with subdomain boundary \tilde{Γ}, such that

$$Ω = {⋃}_{1}^{n} \tilde{Ω}$$

and n is the total number of subdomains that comprise Ω. The unition of interior of subdomain is denoted as

$${Ω}^{′} = \text{?} ( int ) \text{?} ,$$

$$\text{?} \text{indicates text missing or illegible when filed}$$

and the union of subdomain boundaries is

$$\text{?} = {⋃}_{1}^{n} \text{?} .$$

$$\text{?} \text{indicates text missing or illegible when filed}$$

Using the scale split feature of the variational multiscale method for the momentum balance equation and the advection equation, decompose the trial solution fields (u,C) can be decomposed into coarse and fine scales. In the context of nonlinear problems, this decomposition can be viewed as a projection of sub-grid scales onto resolved scales, e.g., for the velocity field u′= [symbol] (u−ū)

$$u ( x , t ) = {\underbrace{\overline{u} ( x , t )}}_{coarse scale} + {\underbrace{{u}^{′} ( x , t )}}_{fine scale} C ( x , t ) = {\underbrace{\overline{C} ( x , t )}}_{coarse scale} + {\underbrace{{C}^{′} ( x , t )}}_{fine scale} \qquad ( 31 )$$

The spaces of coarse scale functions are linearly independent of those of the fine scales, i.e.,

$$\overline{u} ( x , t ) ∈ {\overline{𝒰}}_{t} and {u}^{′} ( x , t ) ∈ {𝒰}_{t}^{′} where {\overline{𝒰}}_{t} ⊕ {𝒰}_{t}^{′} = {𝒰}_{t} and {\overline{𝒰}}_{t} ⋂ {𝒰}_{t}^{′} = ∅ .$$

The same holds for the concentration field. Similarly, the corresponding weighting functions (w,η) can be decomposed as:

$$w ( x ) = \overline{w} ( x ) + \text{?} {w}^{′} ( x ) η ( x ) = \overline{η} ( x ) + {η}^{′} ( x ) \qquad ( 32 )$$

$$\text{?} \text{indicates text missing or illegible when filed}$$

By the additive nature of the decomposition of weighting function in Equation\. (32), the weak form can be split into coarse-scale and fine-scale sub-problems by grouping the terms depending on the weighting functions at either scale:

### Coarse-Scale Sub-Problem

$$( \overline{w} , \text{?} ) + ( \overline{w} , u \cdot ∇ u ) + ( ∇ \overline{w} , {σ}_{v} ( u ) ) - ( ∇ \cdot \overline{w} , p ) = ( \overline{w} , {h}_{M} \text{?} + ( \overline{w} , \text{?} ) \qquad ( 33 )$$

$$( q , ∇ \cdot u ) = 0 \qquad ( 34 )$$

$$( \overline{η} , \text{?} ) + ( \overline{η} , u \cdot ∇ C ) + ( ∇ \overline{η} , κ \cdot ∇ C ) = ( \overline{η} , {h}_{E} \text{?} + ( \overline{η} , f ) \qquad ( 35 )$$

$$\text{?} \text{indicates text missing or illegible when filed}$$

### Fine-Scale Sub-Problem

$$( {w}^{′} , \text{?} ) + ( {w}^{′} , u \cdot ∇ u ) + ( ∇ {w}^{′} \text{?} {σ}_{v} ( u ) ) - ( ∇ \cdot {w}^{′} , p ) = ( {w}^{′} \text{?} ) \qquad ( 36 )$$

$$( {η}^{′} , \text{?} ) + ( {η}^{′} , u \cdot ∇ C ) + ( ∇ {η}^{′} , κ \cdot ∇ C ) = ( {η}^{′} , f ) \qquad ( 37 )$$

$$\text{?} \text{indicates text missing or illegible when filed}$$

### Derivation of Fine Scale Models for Velocity and Concentration Fields

Equations (36) and (37) can be resolved locally and the models for the time dependent fine-scale velocity and concentration fields extracted, via the residuals of the Euler-Lagrange equations of the coarse-scales, respectively. These models when embedded in the coarse scale formulation provide stability in the sense of inf-sup condition as well as high advection velocity. These subgrid models also project missing physics onto the resolved scales. This step restores stability of the mixed weak form and also increases the accuracy of the formulation. The fine-scale sub-problem presented in Equations (36) and (37) yields a nonlinear coupled system. Therefore, the fine-scale variational equations can first be linearized with respect to the fine-scale velocity and concentration fields as follows:

$$( {w}^{′} , \text{?} ) + ( {w}^{′} \text{?} δ {u}^{′} \cdot ∇ \overline{u} ) + ( {w}^{′} , \overline{u} \cdot ∇ δ {u}^{′} ) + ( ∇ {w}^{′} , 2 μ ( \overline{γ} ) ϵ ( δ {u}^{′} ) ) = - ( {w}^{′} , {\overline{r}}_{M} ) \qquad ( 38 )$$

$$( {η}^{′} , δ {C}_{t}^{′} ) + ( {η}^{′} , δ {u}^{′} \cdot ∇ \overline{C} ) + ( {η}^{′} , \overline{u} \cdot ∇ δ {C}^{′} ) + ( ∇ {η}^{′} , \text{?} \cdot ∇ δ {C}^{′} ) = - ( {η}^{′} \text{?} {\overline{r}}_{E} ) \qquad ( 39 )$$

$$\text{?} \text{indicates text missing or illegible when filed}$$

where μ(γ̅) is the nonlinear viscosity that is a function of shear-rate which is calculated based on the coarse-scale velocity field, r̅_{M}=r_{M}(ū,p,C̅) is the residual of the Euler-Lagrange equations of the coarse-scale conservation of momentum, and r̅_{E}=r_{E}(ū,C̅) is the residual of the Euler-Lagrange equation of the coarse-scale concentration equation. These residuals can be defined as:

$${r}_{M} ( \overline{u} , p , \overline{C} ) = \text{?} + \overline{u} \cdot ∇ \overline{u} + ∇ p - 2 μ ( \overline{γ} ) ∇ \cdot ( ϵ ( \overline{u} ) ) - 2 {∇}_{μ} ( \overline{γ} ) ϵ ( \overline{u} ) - \text{?} \qquad ( 40 )$$

$${\overline{r}}_{E} ( \overline{u} , \overline{C} ) = \text{?} + u \text{?} ∇ \overline{C} - ∇ \cdot ( \text{?} \cdot ∇ \overline{C} ) - f \qquad ( 41 )$$

$$\text{?} \text{indicates text missing or illegible when filed}$$

In order to resolve the fine-scale sub-problems (36) and (37), and to derive fine-scale models for the velocity and concentration fields, some simplifying assumptions can be made to localize the problems. The fine-scale trial solutions are assumed to vanish at the subdomain boundaries, namely, u′=0 on Γ′ and C′=0 on Γ. Bubble functions b^{\tilde{Ω}} defined over \tilde{Ω} can be employed to interpolate the fine-scale trial solutions and weighting functions as follows:

$$δ {u}^{′} = \text{?} {w}^{′} = \text{?} δ {C}^{′} = \text{?} {η}^{′} = \text{?} \qquad ( 42 )$$

$$\text{?} \text{indicates text missing or illegible when filed}$$

The above defined fine-scale trial solutions and weighting functions can be substituted into Equations (40) and (41), the mean projection theorem can then be applied, and a backward Euler time marching scheme the used to discretize-in-time the linearized weak forms of Equations (40) and (41). The fine-scale problem can then be resolved, and the projection from the residual of the coarse-scale Euler-Lagrange equations constructed to the fine-scale solution fields V′: {u′, C′}. This provides fine-scale solution in terms of the residuals of the Euler-Lagrange equations scaled by the stabilization tensor

$$[ \begin{matrix}δ {u}^{′} \\ δ {C}^{′}\end{matrix} \text{?} = b [ \begin{matrix}δ {u}^{′} \\ δ {C}^{′}\end{matrix} \text{?} = {\underbrace{- b ( b \text{?} ) {\{ \frac{M}{Δ t} + [ \begin{matrix}\text{?} & \text{?} \\ \text{?} & \text{?}\end{matrix} ] \}}^{- 1}}}_{τ} [ \begin{matrix}{\overline{r}}_{M} \\ \text{?}\end{matrix} \text{?} \qquad ( 43 )$$

$$\text{?} \text{indicates text missing or illegible when filed}$$

where M is the mass matrix and Δt is the time step. For ease of notation, subdomain bubble functions b^{\tilde{Ω}} can be set as b and the superscript can be removed from \tilde{Ω} in the subsequent formulation. The explicit form of various terms in Equation (43) are:

$$\text{?} = \text{?} ( \hat{b} ( u \cdot ∇ b ) + ( b \text{?} {∇}^{T} u + μ ( γ ) {∇}_{b} \cdot {∇}_{b} + μ ( γ ) ∇ b \text{?} ∇ b ) d Ω \qquad ( 44 )$$

$$\text{?} = \text{?} ( \hat{b} ( u \cdot ∇ \text{?} b ) + ∇ b \cdot ( κ \text{?} ∇ b ) ) d Ω \qquad ( 45 )$$

$$\text{?} = \text{?} {\hat{g} ( b )}^{2} d Ω \qquad ( 46 )$$

$$\text{?} = \text{?} {( b )}^{2} \text{?} C d Ω \qquad ( 47 )$$

$$\text{?} \text{indicates text missing or illegible when filed}$$

where τ is the discrete system that relates the fine-scale velocity and concentration fields with the residual of coarse-scale momentum balance and concentration equations. The physical interpretation of \hat{T}_{M} can include the sweeping effect (i.e., the fine-scale velocity transported by the coarse-scale velocity), the distortion effect (i.e., the coarse-scale velocity transported by the coarse-scale velocity), and the fine-scale diffusion; \hat{T}_{E} is the discrete system that relates the fine-scale concentration and the residual of the coarse-scale equation of convection-diffusion equation of concentration transport.

### Stabilized Formulation

The nonlinear coarse-scale sub-problems in Equations (33)-(35) can be linearized with respect to the fine-scale velocity and concentration fields, the resulting formulation combined, and the terms that depend on the fine scale fields grouped.

$$( \overline{w} , \text{?} ) + ( \overline{w} , \overline{u} \cdot ∇ \overline{u} ) + ( ∇ \overline{w} , 2 μ ( \overline{γ} ) ϵ ( \overline{u} ) ) + ( q , ∇ \cdot \overline{u} ) - ( ∇ \cdot \overline{w} , p ) + ( \overline{η} , \text{?} ) + ( \overline{η} , \overline{u} \text{?} ∇ \overline{C} ) + ( ∇ \overline{η} , \text{?} \cdot ∇ \overline{C} ) + ( \overline{w} , \text{?} \overline{C} ) + ( \text{?} ) + ( \text{?} , δ {C}^{′} ) = ( \overline{w} \text{?} + ( \overline{w} , \text{?} ) + ( \overline{η} , {h}_{E} \text{?} + ( \overline{η} , f ) \qquad ( 48 )$$

$$where$$

$$\text{?} = - \text{?} \cdot ∇ w + w \cdot {∇}^{T} u - μ ( γ ) ( ∇ ( ∇ \text{?} w ) + ∇ w ) - ∇ q + η {∇}^{T} C$$

$$\text{?} = \hat{g} w - \text{?} \cdot ∇ η - ∇ \cdot ( \text{?} \cdot ∇ η )$$

$$\text{?} \text{indicates text missing or illegible when filed}$$

By substituting the fine-scale solutions defined in Equation (43) into the corresponding slots in the coarse-scale formulation, a stabilized form is rendered that can expressed in the residual form as follows. Formally stated: Find V=(u,p,C)∈Ut×Pt×Ct, such that ∀W=(w,q,η)∈W×Q×H, the following holds.

$$\text{} \begin{matrix}\text{?} ( w , q , \text{?} u , p , C ) + {ℛ}^{VMS} ( w , q , \text{?} u , p , C ) = \text{?} ( w , η )\end{matrix} \text{} \qquad ( 49 )$$

$$where ,$$

$$\text{?} ( w , q , \text{?} u , p , C ) = \text{?} ( w \text{?} , C ) + \text{?} ( \text{?} u ) + \text{?} ( \text{?} u , C )$$

$${ℛ}^{VMS} ( w , \text{?} u , C ) = ( \text{?} , τ \text{?} )$$

$$\text{?} ( w \text{?} u , C ) = ( w , \text{?} + ( w , \text{?} ∇ \text{?} ) + ( ∇ w , 2 μ ( γ ) ϵ ( \text{?} ) ) - ( ∇ \cdot w , p )$$

$$\text{?} ( q \text{?} u ) = ( q , ∇ \cdot \text{?} )$$

$$\text{?} ( η \text{?} u , C ) = + ( η , \text{?} ) + ( η , u \cdot ∇ C ) + ( ∇ η , \text{?} \cdot ∇ C )$$

$$\text{?} ( w , η ) = ( w , {h}_{M} \text{?} + ( w \text{?} ) + ( η , {h}_{E} \text{?} + ( η , \text{?} )$$

$$\text{?} \text{indicates text missing or illegible when filed}$$

### Circadian Rhythm Modulated Blood Flow

Circadian rhythm is primarily governed by the body's intrinsic timing system, the suprachiasmatic nuclei (SCN) located in the hypothalamus of the brain, which works in conjunction with the light/dark cycle to synchronize the wake/sleep cycle and other physiological processes, including blood pressure. Blood pressure typically follows a predictable pattern over the 24-hour circadian cycle due to heart rate (HR) and systemic vascular resistance (SVR) changes that occur due to the effects of peptides, neurotransmitters, and hormones, which are regulated by the SCN. This pattern typically involves a steep rise in systolic blood pressure (SBP) and diastolic blood pressure (DBP) when awakening in the morning with an afternoon decline, followed by a subsequent decline in SBP and DBP in the evening and into sleep. SCN also influences short-term variations in these signaling molecules, causing short-term blood pressure oscillations over the course of the wake/sleep cycle.

Cell physiology is also regulated by the SCN and exhibits rhythm in cellular metabolism and proliferation, with predictable amplitudes and times of peaks and troughs. These changes can be used to reduce the harmful effects of cytotoxic treatments by administering drugs that target the regulation of cell-cycle events or angiogenesis at specific times (this may be referred to as chronotherapy). Pre-clinical and clinical evidence supports investigations of the chronotherapy hypothesis in cancer patients.

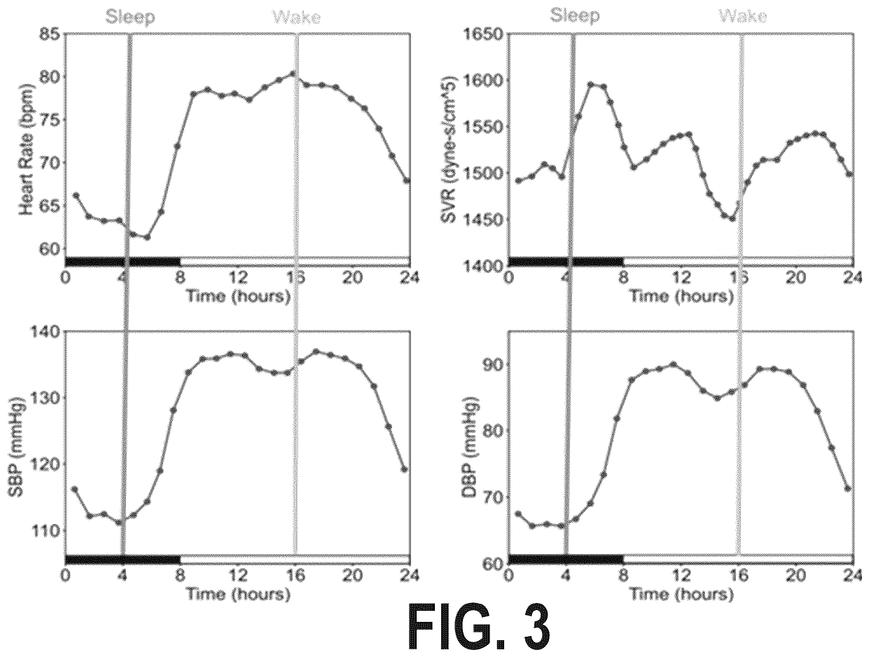

The present disclosure illustrates how the circadian rhythm influences the transport of drug via blood circulation to specific target sites. FIG. 3 shows variations in systolic blood pressure (SBP) and diastolic blood pressure (DBP) in dipper patients who display the typical nocturnal decrease in blood pressure. This decrease in blood pressure correlates with the natural oscillations of heart rate (HR) and systemic vascular resistance (SVR) over the course of the circadian cycle. The clinical data in FIG. 3 was obtained on dipper patients.

FIG. 3 depicts experimental data of dipper subjects showing 24-hour variation of systolic blood pressure (SBP), heart rate (HR), diastolic blood pressure (DBP), and systemic vascular resistance (SVR). The data is organized such that the sleep cycle begins at 12 A.M. and ends at 8 A.M. The magenta and yellow lines show parameters at which sleep and wake cycles are simulated, respectively.

### Experimental Results

Aspects of the methods describe herein were validated on a 3D microfluidic chamber that was lined with C166 mouse endothelium cells at the bottom surface. A solution of PLGA-b-HA nanoparticle with water as a base fluid was injected in the chamber to facilitate particle adhesion. The performance of the models described herein on biologically relevant arterial geometries under in vivo flow conditions was validated. The blood was modeled as a fluid with shear-rate dependent behavior. The resistance from the distal part of the arterial network was modeled via resistance boundary conditions applied at the outflow using Equation (15).

The second and third test cases validated the performance of the method to predict drug transport and adhesion in idealized engineered arteries with bifurcations. The effect of the circadian rhythm modulation of blood flow on nanoparticle adhesion and retention was also investigated. The final test case demonstrated the applicability of the methods described herein to an MRI-based geometry of the carotid artery system, highlighting the effects of curvatures and bifurcations on particle adhesion at target sites.

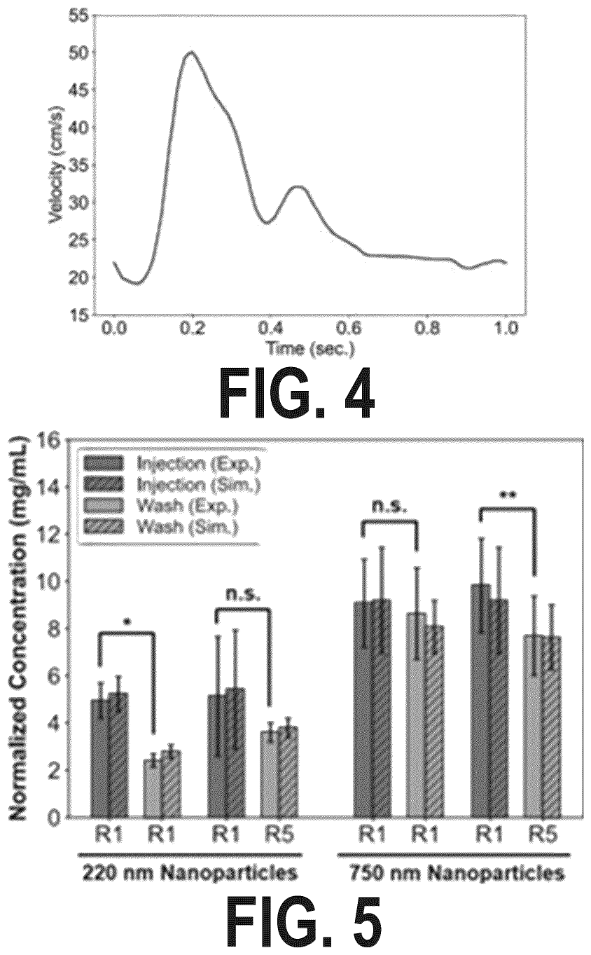

The stabilized finite element method presented in Equation (49) was implemented using equal order, linear and quadratic, tetrahedral and hexahedral elements. The generalized-α method was used for time integration. The nonlinear problem was solved using the Newton-Raphson method and the linear system of equations was solved via the GMRES solver with additive Schwarz preconditioner. Because of the mathematically refined representation of the viscosity field (Equation (12)) in the consistent tangent matrix, the nonlinear solver produced quadratic convergence rates. FIG. 4 shows a velocity waveform in a carotid artery during a typical cardiac cycle that is applied at the inlet to the vascular network. For the engineered bifurcating artery and the idealized arterial tree, the velocity magnitude in FIG. 4 was reduced to obtain shear rates within a range of 10-250 s⁻¹ which are typically observed in microvasculature. The shear rate dependent Carreau-Yasuda model was employed to replicate the non-Newtonian behavior of blood flow, for which the parameters are given in Table 1. Parameters that were used in the adhesion model were experimentally determined and are given in Table 2.

FIG. 4 depicts a velocity waveform in a healthy carotid artery. Time points T_{A} and T_{B} correspond to the maximum and minimum inflow velocities, respectively.

TABLE 2
Parameters for particle-cell adhesion model.

| Parameter | | Value | |
|---|---|---|---|
| 1. | Mean surface density of receptors mᵣ | 10¹³ | #/m² |
| 2. | Surface density of ligands mₗ | 3 × 10¹⁵ | #/m² |
| 3. | Ligand-receptor affinity constant at zero load Kₐ⁰ | 0.069 | μm² |
| 4. | Distance between particles and endothelial cell | 45 | nm |
| | layer at equilibrium Δ | | |
| 5. | Ligand-receptor bond length λ | 5 | nm |

| | | |
|---|---|---|
| 6. | Drag coefficient for spherical particles F^{s} | 1.668 |

### Experimental Validation of the Numerical Method

Microfluidic chamber experiments were carried out to validate the methods described herein. A 3D microfluidic channel of 5×5×30 mm was employed in which endothelial cells that were activated with tumor necrosis factor alpha (TNF-α) to upregulate vascular adhesion molecule (VCAM) expression were attached to the bottom wall. Nanoparticles of poly(lactide-co-glycolide)/hyaluronic acid block copolymer (PLGA-b-HA) conjugated with VHSPNKK peptide, and having varying diameters, were injected into the chamber and particles that attached to the C166 mouse endothelium were imaged. The chamber was then exposed to wash flows with particle-free media at different flow rates that are observed in typical arteries, and the particles that retained on the endothelium were imaged again. This data was used in model validation.

For numerical simulations, the base fluid was chosen as cell culture media, which is an incompressible Newtonian fluid and consistent with what was used in the experiments. A constant parabolic inflow velocity corresponding to a given, but otherwise arbitrary Reynolds number (Re) was prescribed at the inlet wall while stress free boundary conditions were applied at the outlet. No slip velocity boundary conditions were applied on all other walls. Nanoparticles with an average diameter of 220 nm and 750 nm were introduced into the chamber in a time-lagged manner. The process consisted of two stages: a nanoparticle injection stage which was followed by a wash stage with particle-free media. During the nanoparticle injection stage, a 2 mL solution with a nanoparticle concentration of 0.8 mg/mL was injected into the chamber at a flow rate corresponding to the specified Reynolds number. For 750 nm particles, the initial concentration of nanoparticle solution during the injection stage was kept at 0.16 mg/mL. Attachment of nanoparticles to the inflamed endothelial cell layer was simulated by particle-cell adhesion modelled using Robin boundary conditions during the injection stage. In the subsequent wash stage, a 6 mL particle-free fluid was passed through the chamber to study the detachment of the bound nanoparticles. In this stage, the asperity and dispersion models described above were also active in the boundary layer. While particles were injected at Re=1 for all the test cases, washing was done both at Re=1 and Re=5 to study the effect of flow conditions (found in arteries) on retention of the bound particles.

Simulation results matched closely with the in vitro experimental data, as shown in FIG. 5. For comparison of different sets of data, concentration values at the center of the bottom channel wall were normalized w.r.t the concentration of the injected nanoparticles solution. These results validate that the numerical model can accurately predict nanoparticles' adhesion and retention under varying flow conditions, particle sizes, and polymer types.

FIG. 5 depicts a comparative analysis of the adhesion and retention of nanoparticles between numerical simulations and in vitro experiments. The concentration of nanoparticles at the center of the blood vessel-simulating channel bottom was quantified at the end of nanoparticle injection (for adhesion) and after the wash flow with particle-free media (for retention). Particle concentration was normalized w.r.t the concentration of the injected nanoparticle solution during the injection stage. The model described herein was validated for PLGA-b-HA-VHSPNKK particles with an average diameters of 220 nm and 750 nm. R1 and R5 on the x-axis represent Reynolds numbers 1 and 5 of the media flow, respectively. The error bars represent a standard error in each dataset. n.s., *, and ** indicate not statistically significant with p>0.05, p<0.05, and p<0.005, respectively.

### Idealized Bifurcating Artery

The first test case was a three-dimensional engineered bifurcating artery. It consisted of a parent artery where a pulsatile inflow (FIG. 4) corresponding to the flow in a healthy carotid artery was applied. The velocity magnitudes were adjusted to match the average velocity that is found in typical smaller arteries or arterioles. The parent artery was 2 mm in diameter, 12.23 mm in length up to the bifurcation, and 13.95 mm in length to the bifurcation apex. The daughter arteries were both 1.2 mm in diameter and 12.72 mm in length from the bifurcation. The radius of curvature (R_{c}) was 0.1 mm, and the angle between the daughter artery walls was 60 degrees. No-slip boundary conditions were applied at the outer walls of the geometry. Stress-free outflow boundary conditions were applied at the ends of the daughter arteries. The discretization consisted of 33,883 nodes and 21,802 quadratic tetrahedral elements. The time step chosen was 0.01 s.

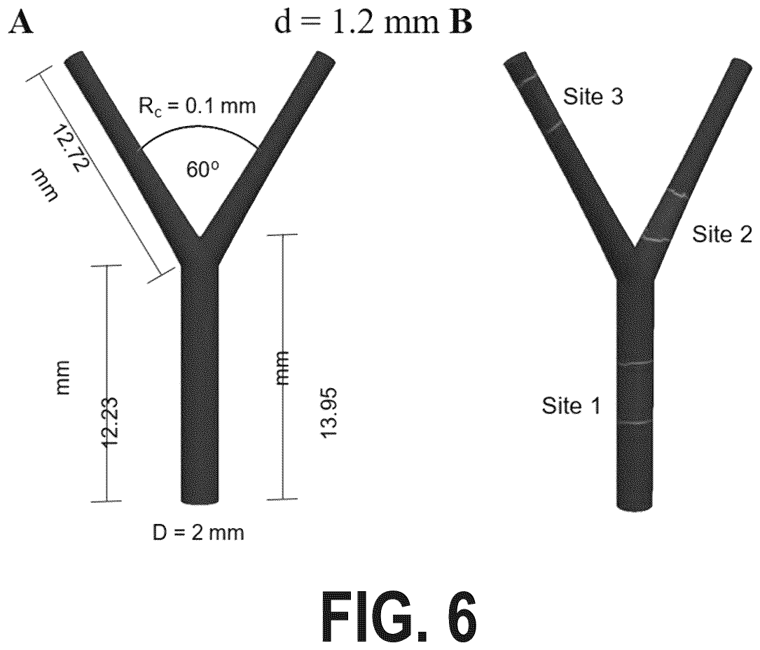

FIG. 6 depicts aspects of a three-dimensional idealized bifurcating artery. (A) Schematic diagram of the idealized bifurcating artery. (B) Three targeted sites, highlighted in red, for drug delivery.

To assess targeted drug delivery, three random sites were specified on the geometry. Site 1 was in the parent artery, while site 2 and site 3 were located near the bifurcation and near the outlet on the two daughter arteries, respectively. The particle-cell adhesion model, asperity model, and dispersion model were active in the boundary layers of these targeted sites. Nanoparticles, characterized by an average diameter of 220 nm and a concentration of 0.1 mg/mL in blood, were introduced into the system at the inlet over a duration of 30 cardiac cycles. Subsequently a wash phase ensued during which blood flow that was devoid of nanoparticles was passed through the geometry for an additional 30 cycles. The wash cycle helped in assessing the extent of drug retention at the targeted sites against the dislodging hydrodynamic forces.

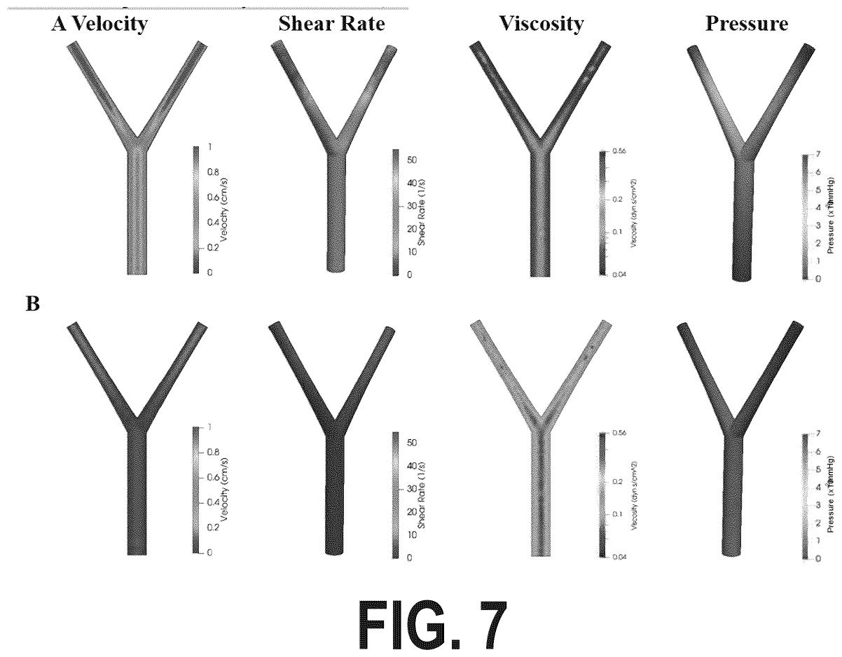

FIG. 7 presents velocity, shear rate, viscosity, and pressure profiles in the bifurcating artery at two distinct time points: T_{A} representing peak systole and T_{B} corresponding to early diastole. At early diastole, the flow fields exhibited significantly reduced values in comparison to peak systole, primarily because this phase coincides with the minimum velocity magnitude on the velocity waveform (see FIG. 4). Additionally, higher flow velocities occurred near the outlets of the two branching arteries due to their smaller diameters, resulting in maximum shear rates along the outer walls adjacent to these outlets. In contrast, the bulk flow at the artery's center exhibited minimal shear rates due to lower velocity gradients, resulting in the highest viscosity in the bulk flow and a decrease in the boundary layer. This viscosity variation followed the shear-thinning blood flow model, with viscosities nearing μ_{∞} during peak systole and returning closer to the initial viscosity μ₀ during early diastole. At the artery's inlet, pressure was at its peak and gradually decreased along the length of artery. The laminar flow ensured there was no significant pressure spike at the bifurcation apex.

The effect of circadian rhythm on nanoparticle adhesion and retention was studied by simulating injection and wash stages during sleep and wake cycles (e.g., as depicted in FIG. 7). Circadian variation in physiological parameters such as inflow velocity, blood pressure, and heart rate were determined from the experimental data presented in FIG. 2 where magenta and yellow lines represent sleep and wake cycles, respectively. Wake cycle has 25% higher inflow velocity, 20% greater pressure, and 27% faster heart rate as compared to the sleep cycle. Since each of the injection and wash stages spans 30 cardiac cycles, a greater heart rate in the wake cycle translated to lesser injection and wash duration. At the end of the injection stage, the concentration of attached particles at target site 1 did not differ significantly between sleep and wake cycles. However, at sites 2 and 3, particle adhesion was 11% and 25% higher, respectively, during the sleep cycle compared to the wake cycle. However, at the end of wash stage, all three target sites showed significantly better drug retention in the sleep cycle as compared to the wake cycle. This is possibly due to the higher pressure and flow rates during the wake cycle that induced stronger dislodging forces. Sites 1, 2 and 3 had 32%, 60%, and 59% higher average particle retention in the sleep cycle as compared to the wake cycle. Time evolution of retention of attached particles during the wash stage is shown in FIG. 7D. The concentration of attached particles during the wake cycles dropped more quickly, as greater numbers of particles were detached under stronger dislodging forces. After 25 seconds, concentration of particles that were retained at the target sites 1 and 2 during the sleep cycle was 23% and 18% higher than the wake cycle, respectively. The study provides evidence that the circadian variation in physiological parameters significantly affects particle retention at the target sites in the blood flow. This test case highlights that the circadian rhythm can be exploited to improve retention time of the nanoparticles and, in turn, improve the efficacy of drug delivery.

FIG. 7 depicts various flow fields in idealized bifurcating artery at (A) peak systole (time point T_{A}) and (B) early diastole (time point T_{B}).

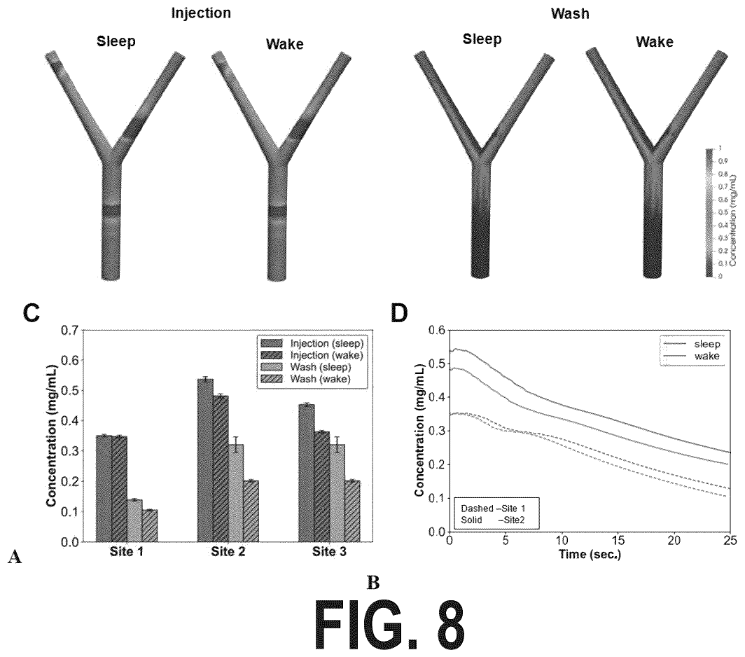

To better understand how the location of the target site within an arterial branch impacts particle adhesion and retention, the time evolution of the mean concentration of particles at sites 1 and 2 is plotted in FIG. 8 for both sleep and wake cycles. Numerical simulations indicated that the concentration at site 1, located in the parent artery, plateaued at 0.34 mg/ml during the injection stage. Any further particle deposition was washed away as the adhesion capacity of the site was maximized. In contrast, the concentration at site 2, located near the bifurcation, continued to increase. At bifurcations, blood flow created regions of disturbed flow that favor nanoparticle accumulation. The formation of low-velocity regions on the inner side of the branch in this case increased the retention of nanoparticles near the vessel walls, promoting their adherence to the target endothelium layer as evidenced in the case of target site 2. During the wash stage, the concentration of particles at the sites decreased as the external flow dislodged some of the attached particles. This behavior is consistent across both wake and sleep cycles. The simulation showed that variations in vessel geometry also affect the hydrodynamic forces experienced by nanoparticles. Optimized particle design can exploit these forces for enhanced targeting and retention in specific regions.

FIG. 8 depicts the effect of circadian rhythm on drug adhesion and retention in an idealized bifurcating artery. Particle adhesion at the three target sites at the end of (A) injection stage, and (B) wash stage is qualitatively shown. (C) Comparison of mean concentration across the three target sites at the end of injection and wash stages. Particle adhesion at sites 2 and 3 was 11% and 25% more in the sleep cycle as compared to the wake cycle, respectively. While negligible variation was observed between the two cycles at site 1, all three target sites showed better drug retention in the sleep cycle as compared to the wake cycle. (D) Evolution of drug retention during wash stage for sleep and wake cycles. After 25 seconds of wash flow, the concentrations of drug particles at sites 1 and 2 were 23% and 18% higher, respectively, during the sleep cycle as compared to the wake cycle.

### Idealized Arterial Tree

Carotid artery stenosis restricts oxygen flow to the brain, which requires a constant oxygen supply for proper functioning. Even a brief interruption in blood and oxygen supply can lead to brain cell damage, which can be triggered within minutes. Severe narrowing or blockage of carotid arteries can result in strokes, and if plaque fragments break off, they can also obstruct brain blood flow, leading to strokes. In this test case, an idealized arterial system with a stenosed branch was used to simulate drug transport at targeted stenosis sites.

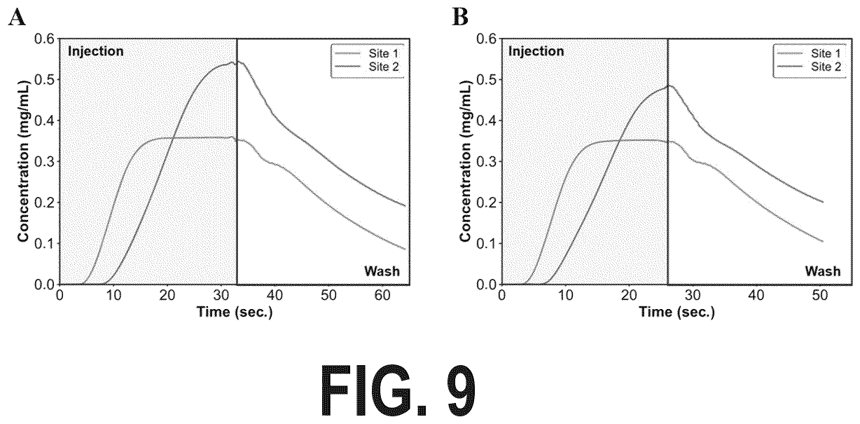

FIG. 9 depicts time evolution of nanoparticle concentration at the target sites 1 and 2 during (A) sleep cycle and (B) wake cycle.

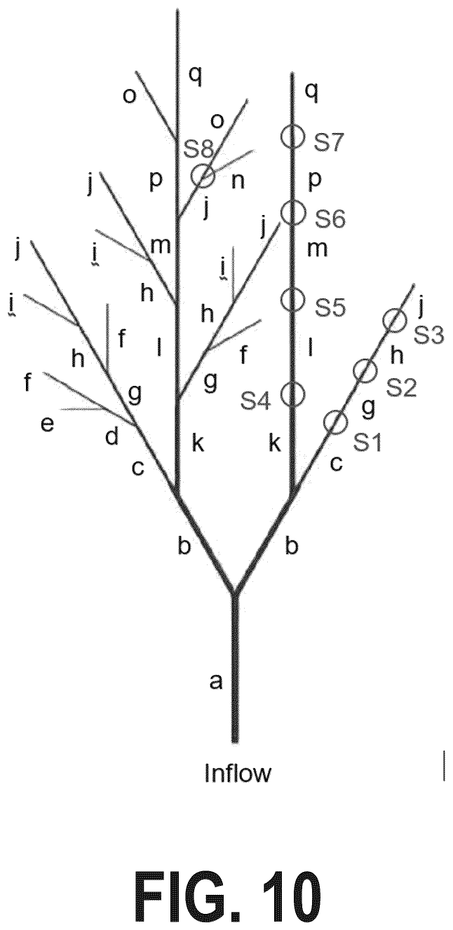

FIG. 10 depicts an idealized arterial system geometry with an inlet at the bottom and multiple outlets. The left side represents a healthy branch with further bifurcations into smaller arteries while the right side represents a stenosed branch with blocked smaller arteries at the bifurcations. Thus, only large branches were constructed on the right side. The drug delivery targets (S1 to S7) are these blocked bifurcations. An additional site S8 in the left branch is activated halfway through the injection stage to simulate particle adhesion to target endothelial cells appearing at bifurcations. The geometric details of each branch are given in Table 3.

TABLE 3
Diameters and lengths of branches in idealized arterial system.

| Label | Diameter (mm) | Length (mm) |
|---|---|---|
| a | 1.26 | 25.2 |
| b | 0.98 | 19.6 |
| c | 0.572 | 11.44 |
| d | 0.334 | 6.67 |
| e | 0.195 | 3.89 |
| f | 0.304 | 6.08 |
| g | 0.521 | 10.42 |
| h | 0.475 | 9.5 |
| i | 0.277 | 5.54 |
| j | 0.433 | 8.66 |
| k | 0.893 | 17.86 |
| l | 0.814 | 16.28 |
| m | 0.742 | 14.84 |
| n | 0.253 | 5.05 |
| o | 0.394 | 7.89 |
| p | 0.676 | 13.52 |
| q | 0.616 | 12.32 |

The geometry of the simulated arterial system is shown in FIG. 10. The diameter at the inlet was 1.26 mm and it symmetrically bifurcated into two daughter branches with diameter 0.98 mm each. The bifurcation angle was 60 degrees. After the first bifurcation, the two daughter branches with a diameter Dₚ asymmetrically bifurcated into a large branch with diameter 0.911Dₚ, and a small branch with diameter 0.584Dₚ. From the second bifurcation onwards, the bifurcation angle was set equal to 60 degrees. Each branch kept the same diameter (i.e., Dᵢ) along a length, 20Dᵢ. The healthy branch (i.e., the left branch in FIG. 10) was constructed by following the above geometric ratios. For the stenosed branch (i.e., the right branch in FIG. 10), the smaller downstream arteries were ‘blocked’ at the bifurcations. Consequently, only the large branches were constructed, while maintaining the same change in the diameters at the corresponding bifurcation locations as in the healthy branch. The drug delivery targets were precisely these blocked smaller arteries at the bifurcations, as indicated in FIG. 10. Table 3 presents the diameters as well as lengths of all the branches of the full geometry shown in FIG. 10.

The geometry was discretized into 217,365 quadratic tetrahedral elements with 127,144 nodes. The problem was run with a time step of 0.01 s. A parabolic velocity profile was prescribed at the inflow with an average velocity of 3 mm/s following the typical waveform as given in FIG. 4. Stress-free velocity boundary conditions were applied at the outlets while no-slip velocity boundary conditions were applied at all other boundary walls. For the pressure field, resistance boundary conditions at the two outlets were applied such that the pressure at the outlet is 120 mmHg during systolic and 80 mmHg during diastole.

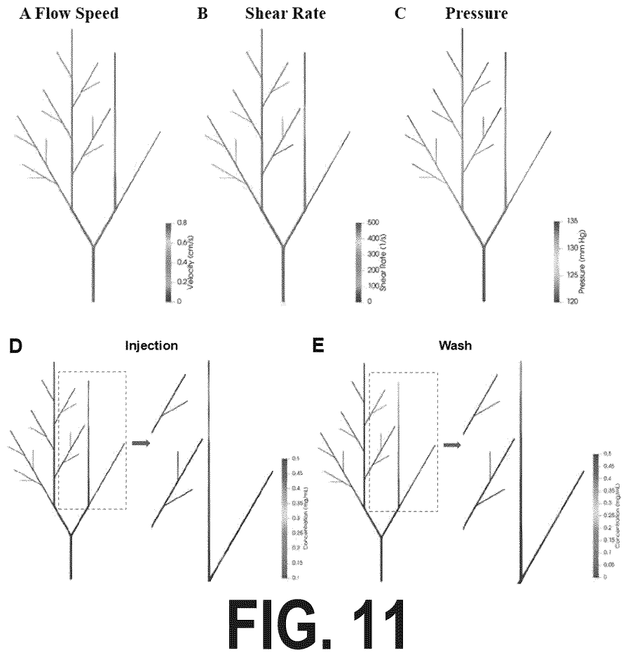

FIG. 11A shows the velocity magnitude contours in the arterial tree. Since the stenosed branch had larger downstream resistance as compared to the healthy branch due to its blocked smaller arteries, the healthy branch (i.e., left bottom portion in FIG. 11A) showed larger velocity magnitude than in the stenosed branch (i.e., right bottom portion in FIG. 11A). A similar pattern was observed for shear rates that were higher in the upstream healthy branch as compared to the stenosed branch. Further downstream in the healthy branch, the flow velocity reduced in the smaller arteries. However, the opposite behavior was observed in the stenosed branch where smaller arteries had higher shear rates due to the blocked bifurcations.

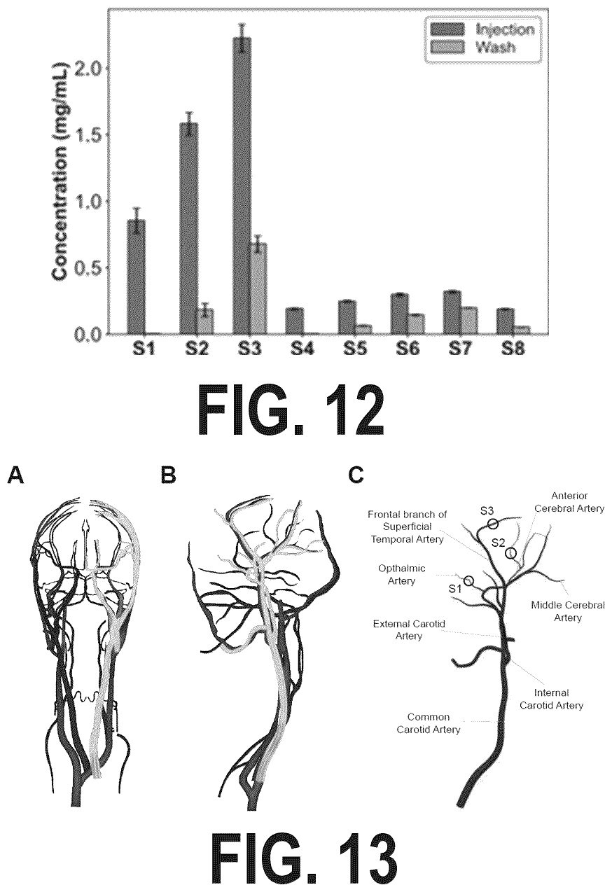

In order to transport ligand-coated particles to the target stenosis sites, nanoparticles with an average diameter of 220 nm were injected into the bloodstream at the inlet using a solution with a concentration of 0.1 mg/mL. Attachment of nanoparticles to the stenosis sites was governed by particle-cell adhesion model, implemented via Robin boundary conditions. FIG. 11D shows a concentration profile of particles at the end of the injection stage. At arterial bifurcations, the blood flow exhibited local variations in flow velocity and shear stress, which are critical in determining endothelial cell behavior and function. These hemodynamic changes can lead to altered endothelial cell responses and are associated with regions that are prone to atherosclerosis due to low or oscillatory shear stresses. To simulate this phenomenon, an additional target site S8 was introduced in the healthy left branch which was activated halfway during the injection stage. Concentration buildup due to the attached particles was seen at all the target sites. In the stenosed branch, a bulk of drug went into the sub-branch with target sites S1 to S3 resulting in the maximum concentration of attached particles at those sites (FIG. 12). In the subsequent wash stage, a particle-free blood flow was maintained for another 30 cardiac cycles. Almost all of the particles were washed away at sites S1 and S4, while 12%, 30%, 26%, 48%, 62%, and 17% of the particles were retained at sites S2, S3, S5, S6, S7, and S8, respectively at the end of wash stage. Site 3 showed maximum adhesion and retention of nanoparticles.

FIG. 11 depicts spatiotemporal dynamics of particle transport, attachment, and retention in an idealized arterial tree with targeted stenosis sites. (A) Velocity magnitude contour at peak systole T_{A}. (B) Shear rate at peak systole T_{A}. (C) Pressure profile at early diastole T_{B}. (D) Concentration profile at the end of injection stage, and (E) wash stage. Concentration build-up can be observed at the targeted sites (S1 to S7) in the stenosed branch as well at site S8 in the healthy left branch. The concentration of particles then dropped down at the end of the wash stage.

FIG. 12 depicts a comparison of mean concentration across all the target sites in the idealized arterial tree at the end of the injection and wash stages.

### In Vivo Carotid Artery System

In this test case, the drug delivery computational framework with non-Newtonian blood flow model was applied to an in vivo geometric model obtained from MRI scans as shown in FIG. 13. A computational grid was superimposed on a patient-specific 3D model of the carotid arterial system, consisting of 388,508 nodes and 243,839 quadratic hexahedral elements. The computational geometry included the common carotid artery, which ascends within the neck and bifurcates into the internal and external carotid arteries. The two target sites, S1 and S2, were located in the ophthalmic artery and anterior cerebral artery, respectively, which branch off from the internal carotid artery. The anterior cerebral artery supplies blood to the medial portions of the primary motor and sensory cortices, and any blockage can lead to a stroke affecting leg motor and sensory functions. Target site S3 was situated in the frontal branch of the superficial temporal artery, which supplies blood to the scalp and forehead tissue. The anatomy of the computational geometry along with the three target sites is illustrated in FIG. 13.

FIG. 13 depicts an in vivo geometric model of the arterial system in the brain along with the superposed computational grid (highlighted in yellow). (A) Frontal view (B) Side view (C) Anatomy of modeled geometry with target sites.

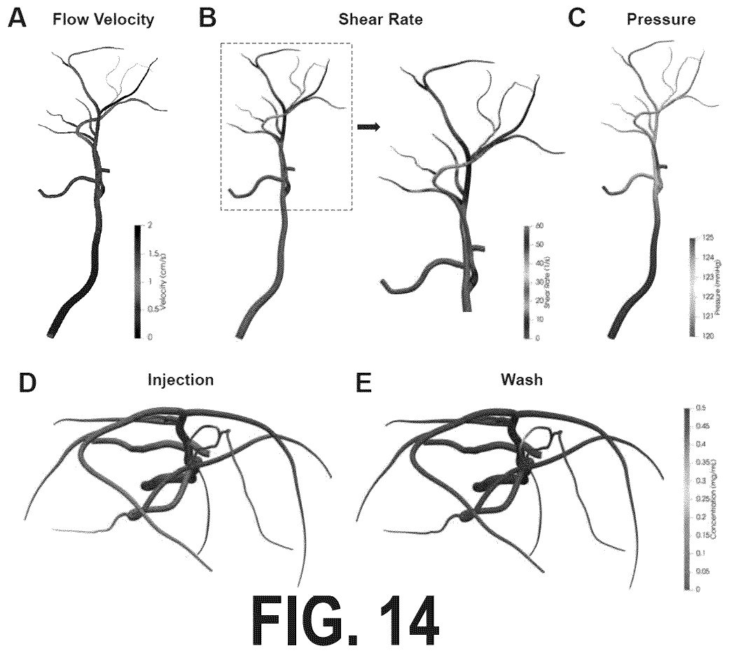

A physiologically relevant pulsatile inflow velocity of 35 cm/s was applied at the inlet located in the common carotid artery. No-slip velocity boundary conditions were applied at all the vessel walls. The resistance boundary condition in equation (15), which produces a physiological pressure waveform at the outlets, was imposed to incorporate the downstream resistive effects of the smaller branching arteries. The resistance parameter R for each branch was tuned based on the flow rate in the branches, calculated with traction-free boundary conditions, to obtain a pressure amplitude between 85-125 mmHg at the outlets. The simulation was run with a time step of 0.01 seconds. The coupled system of equations converged quadratically in the nonlinear iterative solution procedure at each time step. The pressure and velocity decreased sharply as blood flowed into the smaller branches of the external and internal carotid arteries, as shown in FIG. 14(A-C). The shear rates in the smaller branches where the target sites are also located ranged between 10-100 s⁻¹, which is typical for microvasculature. FIG. 14A presents a volume rendering of the magnitude of the velocity field. FIG. 14(B-C) shows instantaneous snapshots of the shear rate and pressure field at peak systole, where these quantities reached their maximum value due to higher blood velocity. The anatomical features, including the curvature and geometry of blood vessels, influenced various aspects of the blood flow, such as velocity profiles and shear stress distribution. Shear stress exhibited greater fluctuations at locations of curvature and bifurcations (FIG. 14B), which in turn influenced drug adhesion and retention.

FIG. 14 depicts flow physics in the carotid artery system at peak systole. (A) flow velocity, (B) shear rate, and (C) pressure. (D) Nanoparticles that were injected at the inflow were attached at all the target sites as shown by the buildup of nanoparticle concentration at the end of the injection stage. The maximum buildup was in the anterior cerebral artery. (E) A fraction of the attached particles were dislodged under the effect of hydrodynamic forces during the wash stage.

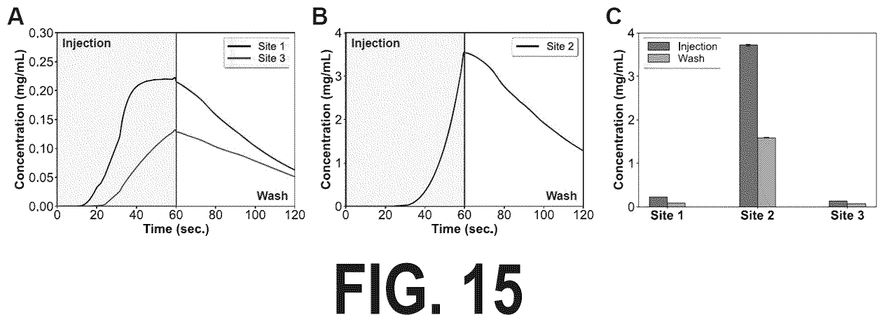

For drug delivery, nanoparticles were injected at the inlet for 60 cardiac cycles. The particles were transported to the target sites by the flow of blood, where they attached under the influence of particle-cell adhesion forces. FIG. 15(A-B) shows the time evolution of the mean concentration at the three target sites. Adhesion and retention of particles at the anterior cerebral artery (site 2) were an order of magnitude higher compared to the other two sites located in the ophthalmic artery and the frontal branch of the superficial temporal artery. This highlights the significant impact of the local geometric shape as well as the location of the target site on particle adhesion and retention. Site 2, located at a curvature of the blood vessels, experienced non-uniform shear stress distribution, with areas of lower shear stress typically found on the inner wall of the curve. These areas provided a favorable environment for nanoparticle accumulation due to reduced shear forces acting on the particles, allowing them to remain in contact with the vessel wall for longer periods. Adhesion at site 1 saturated after 40 seconds, resulting in a plateau in the time evolution plot (FIG. 15A). Site 2 showed an approximately linear increase in the concentration of attached particles, while site 3 showed an exponential increase in particle concentration. After the injection, wash stage ensued for another 60 cardiac cycles. During the wash stage, the concentration drop was most rapid at site 1. At the end of the wash stage, 36%, 43%, and 48% of particles were retained at target sites 1, 2, and 3, respectively (FIG. 15C). The simulation described herein provides an understanding the impact of vessel curvature and geometry on patient-specific in vivo models with geometric uncertainties, allowing for the strategic design of nanoparticles that preferentially accumulate in target areas, ensuring localized and controlled drug release.

FIG. 15 depicts time evolution of mean concentration at (A) target sites 1 and 3, as well as (B) target site 2, during the injection and wash stages. Adhesion and retention of particles at the anterior cerebral artery (site 2) was an order of magnitude higher as compared to the other two sites. (C) Comparison of mean concentration across all the target sites at the end of injection and wash stages. At the end of wash stage, 36%, 43%, and 48% particles were retained at target sites 1, 2, and 3, respectively.

A computational model is described herein to investigate nanoparticle-based targeted drug delivery under the influence of circadian rhythm-modulated blood flow in virtual in vivo arterial geometries. This model provides an understanding of how complex arterial geometries and physiological changes over the circadian cycle affect the transport, adhesion, and retention of nanoparticles within the vasculature, allowing for optimization of the design of such nanoparticles and the timing of their delivery so as to improve the deposition and retention of such nanoparticles at target sites within a patient. This computational framework integrates a shear-rate-dependent non-Newtonian blood flow model with drug adhesion models, accounting for the complexities of nanoparticle transport in realistic arterial geometries that are derived from MRI scans.

The simulations incorporate various factors influencing nanoparticle behavior, including vessel curvature, bifurcations, and downstream resistance, to provide a comprehensive understanding of drug delivery dynamics. The experimental results demonstrate that vessel geometry and location significantly impact nanoparticle adhesion and retention. Curved regions and bifurcations, characterized by non-uniform shear stress distributions, create favorable conditions for nanoparticle accumulation, highlighting the importance of target site selection for optimized drug delivery.

Circadian variations in physiological parameters, such as blood pressure and flow velocity, were shown to influence nanoparticle retention. During the sleep cycle, lower shear forces and flow rates resulted in higher nanoparticle retention as compared to that during the wake cycle. This finding underscores the utility of chronotherapy to enhance drug delivery efficacy by aligning treatment schedules with the body's natural circadian rhythms. The computational framework described herein, validated through in vitro experimentation, can be used as a highly accurate virtual platform to provide comprehensive insights into the variation of hemodynamic parameters across patient-specific in vivo models in response to physiological changes in blood flow and vessel geometries. These simulations also show that local variations significantly affect particle adhesion and retention at target sites. By leveraging these insights, the design of nanoparticles the timing of their provision to a patient can be enhanced to preferentially accumulate in target areas.

### ADDITIONAL EXAMPLE EMBODIMENTS AND EXPERIMENTAL RESULTS

Drug delivery via nanocarriers can provide increased delivery efficacy while reducing off-target effects, thereby enhancing therapeutic outcomes. Drug carrying nanoparticles can be tethered with peptides or antibodies bound to the surface of drug encapsulating nanoparticles. Computational Fluid Dynamics (CFD) methods can provide insights into the effects of circadian rhythm, nanoparticle configuration, or other factors on the drug-nanoparticle transport, distribution, and interactions with the vascular and interstitial environments. Embodiments described herein include an advanced fluid dynamics modeling method that is based on convective transport of viscous incompressible fluid (blood), coupled with a scalar advection diffusion equation for the formation of drug concentration gradients within the transport fluid domain and buildup of nanoparticle concentrations at the targeted site(s). These embodiments can include an experimentally calibrated particle-endothelial cell adhesion model, a friction model accounting for surface roughness of endothelial cell layer, and a dispersion model for particle transport in the boundary layer. These specific methods provide highly accurate representations of the relevant processes across widely disparate temporal and spatial scales while reducing the computational cost (e.g., with respect to processor cycles, memory, storage, network interconnect bandwidth, power, or other costs of computation) to obtain such accurate simulations. Comparison with microfluidic experiments validate the predictive capability of these mathematical models for drug transport, adhesion, and retention at the target site(s).

Nanoparticles tethered with vasculature-binding epitopes have been used to deliver drug into injured or diseased tissues via the bloodstream. The extent that blood flow dynamics affect nanoparticle deposition and retention at the target site after adhesion was previously poorly quantified. Such a knowledge gap potentially underlies previously observed significant differences in therapeutic efficacy between animal models and humans. Embodiments described herein include an experimentally validated mathematical model that accurately simulates the effects of blood flow on nanoparticle adhesion and retention, thus overcoming the limitations of conventional trial-and-error-based drug design in animal models. These embodiments include an integrated mathematical method that can include a combination of a mechanics-based dispersion model for nanoparticle transport and diffusion in the boundary layers, an asperity model to account for surface roughness of endothelium, and an experimentally calibrated stochastic nanoparticle-cell adhesion model to describe nanoparticle adhesion and subsequent retention at the target site under external flow. PLGA-b-HA nanoparticles tethered with VHSPNKK peptides that specifically bind to vascular cell adhesion molecules on the inflamed vascular wall were experimentally investigated. The computational model described herein revealed that larger particles perform better in adhesion and retention at the endothelium for those particle sizes suitable for drug delivery applications and within physiologically relevant shear rates. The computational model described herein also corresponded closely to the in vitro experiments, demonstrating the utility of such models with respect to improving the design of nanocarriers for vascular microenvironments, thereby substantially reducing in vivo experimentation as well as the development costs. Further, the significantly reduced computational cost associated with the embodiments described herein facilitate their use on a per-patient basis, designing or selecting improved nanoparticle configuration (e.g., with respect to size, target or binding agent used, geometry) and/or dose timing (e.g., relative to a specific patient's circadian rhythm or pattern of blood pressure variation over the day) for individual patients based on their specific anatomy, blood pressure, circadian rhythm, etc.

In some drug delivery embodiments, drug molecules can be encapsulated within biodegradable nanoparticles tethered with bioactive ligands that are capable of binding to receptors that are overexpressed in the microvascular networks of target tissues (e.g. tumors). Through the interaction of ligands and respective binding proteins, these nanoparticles can deliver their drug cargo continuously or in response to stimuli, thereby improving the bioavailability of the drugs at target sites. Prior attempts to quantify the deposition of such nanoparticles in real anatomy via simulation have been limited in terms of their predictive capability for patient-specific attributes and demand significant computational resources. Mathematical modeling methods as described herein can accurately simulate the effects of size, shape, biochemical properties, and mechanical stiffness of nanoparticles on their transport and adhesion in biological systems, and for significantly reduced computational cost relative to alternative methods.

Prior attempts at simulation of such systems have failed to account for the detachment of attached particles under the influence of external flow. The in vitro studies described herein provide experimental evidence that the concentration of PLGA-b-HA particles adhered to a target vascular wall decreases over time due to the dislodging forces exerted by blood flow. This effect is accounted for in the various simulation methods describe herein through a coupling between the adhesion force engendered via the ligand-receptor bond that appears in the probability function in Decuzzi's adhesion model, the dislodging mechanical force field induced via external flow, and shear-induced diffusion in the boundary layer. Consequently, the numerical methods described herein accounts for the nonlinear hydrodynamic forces on the nanoparticles through a combination of computationally low-cost mechanics-based models. A comprehensive framework that can model and accurately predict the intricate relations between various physiological effects and dislodging forces induced by blood flow is described herein. These embodiments are able to highly accurately predict the retention time of the nanoparticles and, in turn, improve the efficacy of drug delivery.

The physical modeling of microvascular transport and adhesion of nanoparticles can be broadly categorized into discrete, hybrid, and continuum models that are operational at the molecular scale, nanoscale, and microscopic/mesoscopic length scales, respectively. Continuum models provide a computationally robust way to simulate drug particles over arterial length scales by integrating binding kinetics information from the nanoscale. The finite element method (FEM) is the most commonly used technique for continuum drug delivery simulations. Nanoparticles can be represented as a homogenized mixture in the fluid and an advection-diffusion equation can be used to simulate the formation of drug concentration gradients in the blood flow.

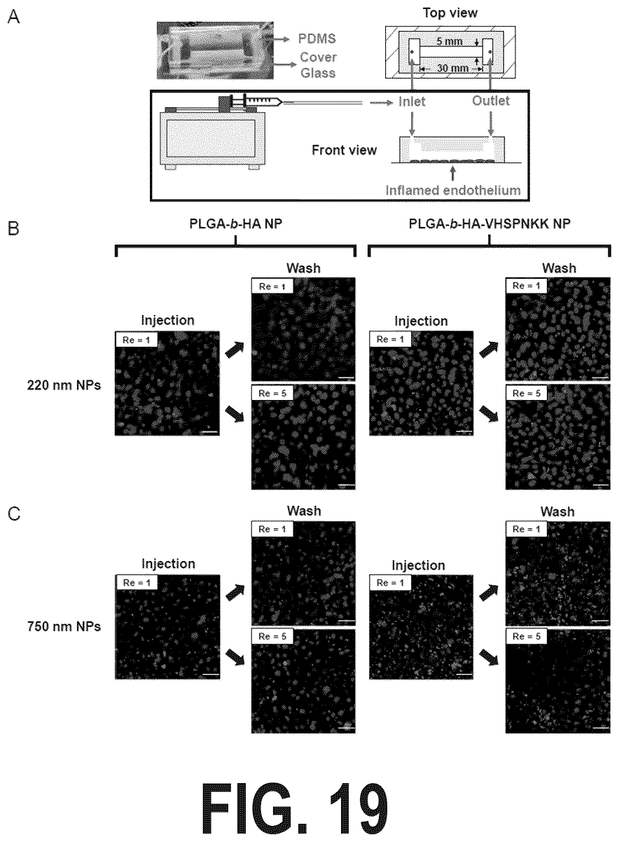

Embodiments described herein include a specific integration of the Taylor-Aris dispersion model with an asperity model with Decuzzi's particle-cell adhesion model. The dispersion model accounts for shear-induced diffusion of particles in the boundary layer, while the asperity model accounts for the interaction between the fluid and endothelial cell layer. Prior attempts at modeling such systems have failed to account for detachment of the attached nanoparticles is. This is an important factor for accurately predicting nanoparticle-based drug delivery, as it influences the available drug for uptake. The experiments described herein illustrate that not all nanoparticles stay attached to the endothelium after injection (FIGS. 19 and 10). Some of the particles were detached under the action of dislodging hydrodynamic forces. Therefore, the assumption of firm adhesion (which is used by prior methods which fail to account for particle detachment) does not remain valid in practical applications. Since the dislodging hydrodynamic force in the adhesion model of prior models is based on the assumption of linear shear flows, it can not take into account the nonlinear inertial and convective forces of real-world intravascular flow fields. In the present disclosure, the adhesion model is complimented with a mechanics-based asperity and Taylor dispersion models to predict nanocarrier transport, attachment, and detachment with enhanced accuracy.

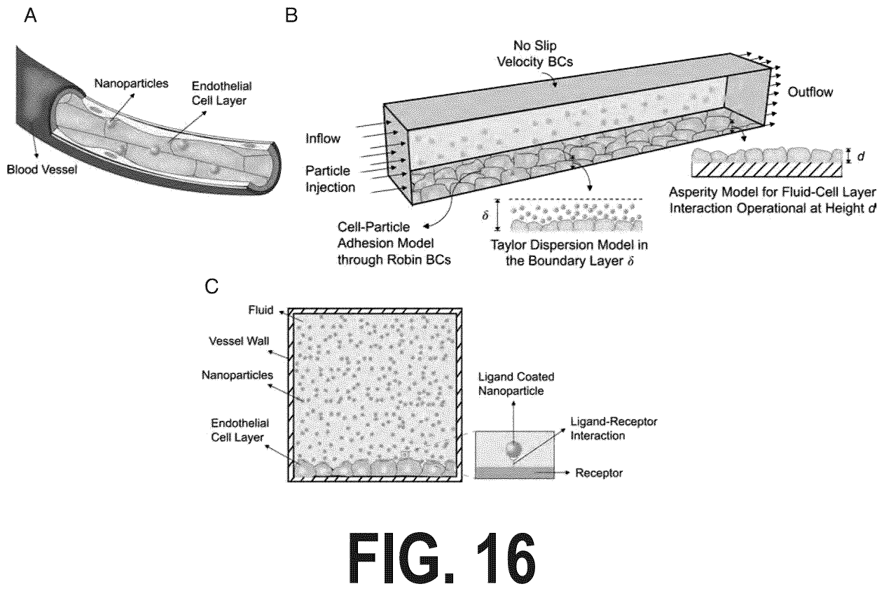

The computational framework described herein can be implemented using a stabilized finite element method to simulate spatiotemporal dynamics of nanoparticles in blood vessels (FIG. 16). A Newtonian rheological model for fluid flow was employed to model the fluid media used in the experimental setup. The particle-cell adhesion model was implemented via a Robin boundary condition (BC) at the endothelial cell layer surface. Robin BC combines Neumann (drug flux in normal direction) and Dirichlet (drug concentration) boundary conditions through a coefficient referred to herein as the vascular deposition parameter. This parameter accounts for particle-cell binding kinetics by integrating information about biochemical, biophysical, and geometric properties from the micro/nanoscale into the macroscale, resulting in significant computational cost savings while maintaining simulation accuracy. The method described herein was validated via 3D microfluidic in vitro experiments.

Microfluidic chamber experiments were carried out wherein C166 mouse endothelial cells were activated to up-regulate vascular adhesion molecule (VCAM) expression. Only the bottom plate was coated with cells to facilitate imaging of fluorescent nanoparticles attaching at the target endothelium to validate the computational model. Poly(lactic-co-glycolic acid)-blockhyaluronic acid (PLGA-b-HA) nanoparticles were chosen over conventional PLGA nanoparticles to mitigate the protein corona formation fouling effects of increased size. Hyaluronic acid grafted to PLGA provided resistance to protein corona formation due to a higher negative zeta-potential than pure PLGA particles. Particles of varying diameters (220, 750, 1,000 nm), both with and without VHSPNKK peptide conjugation, were injected into the chamber and particles that attached to the endothelium were imaged. The size range of the particles was chosen to correspond to particles commonly used in drug delivery applications. Particle systems >1 μm diameter can be problematic due to vascular blocking and particle systems <100 nm diameter are usually used in applications such as biosensing/bioimaging. The chamber was then exposed to wash flows with particle-free media at shear rates ranging from 1 to 250 s⁻¹, which are typical physiological shear rates observed in microvasculature. The particles that were retained on the endothelium were imaged again. These data were used in model validation and to investigate the intricate interplay between various competing mechanisms that have been reported in the in vitro experiments

### Spatiotemporal Modeling of Ligand-Coated Particle Transport, Adhesion, and Retention Over Endothelium

A continuum-based approach was adopted to simulate blood flow, particle transport, and particle-cell adhesion within a vascular channel. The continuum hypothesis and Newtonian model were employed as accurate approximations for the in vitro flow model of ligand-receptor binding in sufficiently larger vessels under laminar flow conditions. The physics-based model for incompressible Newtonian fluid flow and particle transport in the vascular domain Ω is given by the Equations (1)-(3) above. The mathematical model was supplemented with the initial conditions in the domain x∈Ω at time t=t0 and boundary conditions on the domain boundary Γ given in Equations (4)-(10) above.

### Particle-Cell Adhesion Model

FIG. 16 depicts aspects of an experimentally validated computational framework for drug carrier transport, adhesion, and retention in blood vessels as described herein. (A) Schematic diagram of drug delivery using nanocarriers designed for active targeting in circulation. (B) In vitro microfluidic chamber for experiments and simulations. For particle adhesion, a Robin boundary condition embedded with a particle-cell adhesion model was employed at the bottom wall, which was lined with tumor necrosis factor alpha (TNF-α)-treated endothelial cells that overexpressed VCAM. The asperity model, as well as the Taylor dispersion model, were operational in the boundary layer to simulate cell surface roughness and dispersion of entrapped particles, respectively. (C) Cross-sectional view of the flow chamber showing ligand-receptor interaction at the bottom cell surface. The ligand-receptor interaction was modeled using the particle-cell adhesion model that couples adhesion forces to binding affinity. In the model, VCAM was used as a receptor on the activated endothelial cell, and VHSPNKK peptide was used as a VCAM-binding ligand.

Since a continuum modeling approach was adopted using the finite element method, information at the micro- and nanoscale as embedded in the computational model via the Robin boundary condition that as applied at the targeted vessel wall Γ_{wall}, as in Equation 16. The Robin boundary condition accounted for particle-cell adhesion based on the probability of adhesion Pₐ. This probability is related to the strength of adhesion: the larger is the value of Pₐ, the larger the adhesive strength of the particle to the endothelial cell layer. Only particles in the boundary layer of the fluid-filled vessel were simulated to experience the binding affinity (FIG. 16C). Thus, Eq. 16 integrates information from the macroscale (vessel geometry and flow conditions) with data from the micro- and nanoscale (particle geometry, receptor density, and affinity), thereby avoiding massive, computationally inefficient discretization over multiple spatial and temporal scales. Additionally, the mass flux of particles in the direction normal to the wall can be related to the local increase in mass of particles adhering per unit surface φ((x,t), as in Equation 17 above.

The probability of adhesion Pₐ includes 2D binding characteristics of receptors and ligand-coated particles that are attached to surfaces, whose bonds are subjected to dislodging hydrodynamic forces. An experimentally validated closed-form expression was employed to account for the probability of adhesion. This expression describes cellular adhesion by coupling bond forces to the binding affinity. The probability of adhesion for ellipsoid particles is given as Equation 18 above, with similar computations used to obtain the various parameters thereof.

### Modeling the Surface Roughness of the Endothelial Cell Layer

The effects of the surface roughness of the endothelial cell layer can be accounted for as described above, e.g., in relation to Equations (20)-(23).

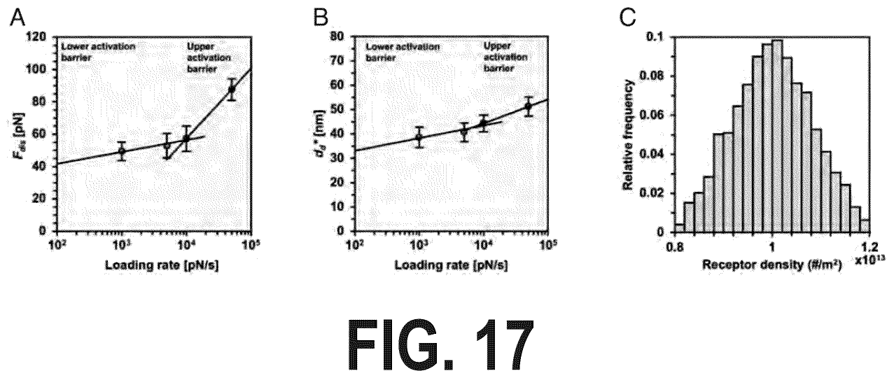

FIG. 17 depicts aspects of the AFM characterization of detachment force and distance for a single ligand-protein interaction and prediction of protein surface density. (A) Average ^{F}dis for 1,000 pN/s, 5,000 pN/s, 10,000 pN/s, and 50,000 pN/s loading rates. The error bars represent 95% CI for each experiment group. (B) Average ^{d}d* for 1,000, 5,000, 10,000, and 50,000 pN/s loading rates. The error bars represent 95% CI for each experiment group. (C) Normal distribution of the surface density of VCAM-1 receptor molecules around its mean value. To account for the nonuniform spatial distribution of cells at the bottom wall, receptor densities were stochastically chosen from the normal distribution.

### Dispersion of Particles in the Boundary Layer

The effect of the dispersion of particles in the boundary layer can be accounted for as described above, e.g., in relation to Equation (24).

### Stabilized Finite Element Method

A variety of methods can be used to simulate the nanoparticle-endovascular systems described herein, e.g., according to the Equations above or similar. For example, the variational multiscale method described above (e.g., in relation to Equations (25)-(49) above) could be employed, or some alternative finite element solver could additionally or alternatively be employed.

### Experimental Results

AFM. In order to strengthen the accuracy of the adhesion model, AFM was used to characterize the interaction strength between VHSPNKK peptide and VCAM1, by generating force-displacement curves at four different loading rates: 1,000, 5,000, 10,000, and 50,000 pN/s. From the retraction traces, the detachment force (F_{dis}) and the detachment distance (d_{d}*) between VHSPNKK and VCAM-1 were characterized. The discernable force jumps observed in the trace experiments indicate an interaction between VHSPNKK and VCAM-1. To minimize the occurrence of multiple ligand-protein binding on a single trace and a greater than 85% certainty that the measured force is a result of a single binding event, the percentage of scans resulting in a noticeable detachment force was targeted to be under 30% by modulating the conjugation of VHSPNKK to the AFM cantilever tip. A detachment-positive trace was observed between 34% and 37% for all loading rates, indicating that the majority of events were due to a single binding event. As such, some of the larger detachment forces seen in the histograms (forces that are 2× greater than the average) may correspond to multiple detachment events per trace. Accordingly, these data were removed from the average detachment force calculations. The experimental results indicate that for increasing loading rates, both the average F_{dis} and average d_{d}* increase (FIGS. 17A and B). This observation corresponds to Bell's theoretical framework that states a molecular complex overcomes an activation barrier before dissociation and hence the dissociation rate positively scales with the pulling force. For the VHSPNKK-VCAM-1 complex the detachment force was found to be on the order of 10 pN for a single interaction at loading rates on the order of 10³ to 10⁴ pN/s. Note that the slope of the detachment force and loading rate relationship abruptly changed for the 50,000 pN/s loading rate experiment. This observation can be explained by the existence of two different energy barriers in the VHSPNKK-VCAM-1 complex. With these experimental observations, a detachment force for a VHSPNKK-decorated nanoparticle bound to VCAM-1 protein can be defined that scales according to the surface density of peptides (mₗ) on the PLGA-b-HA nanoparticles (NPs). Additionally, how this detachment force changes based on the observed shear stress of the nanoparticle can be related to the loading rate.

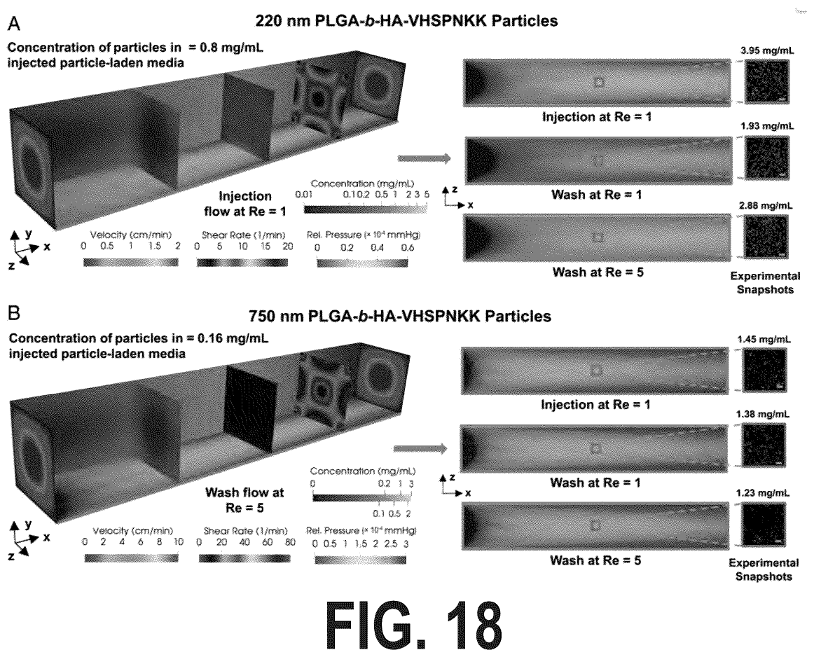

Chamber Flow Simulations. A 3D microfluidic channel of 5×5×30 mm with a tumor necrosis factor alpha (TNF-α) induced inflamed endothelial cell layer attached to the bottom wall was simulated using the numerical methods described herein (FIG. 18 and Table 4). The base fluid was chosen as cell culture media, which is an incompressible Newtonian fluid and is consistent with what was used in the experiments. A parabolic inflow velocity corresponding to a given but otherwise arbitrary Reynolds number (Re) was prescribed at the inlet wall while stress free boundary conditions were applied at the outlet. No slip velocity boundary conditions were applied on all other walls. Nanoparticles with average diameter of 220 nm were introduced into the chamber in a time-lagged manner. The process consisted of two stages: a nanoparticle injection stage which was followed by a wash stage with particle-free media. During the nanoparticle injection stage, a 2 mL solution with a nanoparticle concentration of 0.8 mg/mL was injected into the chamber at a flow rate corresponding to the specified Reynolds number. Attachment of nanoparticles to the inflamed endothelial cell layer was governed by the particle-cell adhesion model via Robin boundary conditions during this stage. In the subsequent wash stage, a 6 mL particle-free fluid was passed through the chamber to study the detachment of the bound nanoparticles. While particles were injected at Re=1 for all the test cases, washing was done both at Re=1 and Re=5 to study the effect of flow conditions found in arteries on retention of bound particles.

FIG. 18 depicts chamber flow simulation results. (A) The probabilistic particle-cell adhesion model results in nonuniform distribution of 220 nm PLGA-b-HA-VHSPNKK particles on the channel bottom wall at the end of the injection and wash stages. The experimental snapshots were taken at the center of the bottom wall and compared with numerical results. Green spots indicate bound nanoparticles over the blue endothelial cells. (B) Numerical simulations showing concentration of 750 nm PLGA-b-HA-VHSPNKK particles in the flow chamber. Larger particles showed a greater retention rate after the wash stage due to larger surface area leading to enhanced adhesion capability with the receptors. Shear rate was maximum in the boundary layer and at the center of the bottom wall, while it was minimum in the bulk flow and along the corners of the chamber. This may also explain the particle distribution pattern in simulation, as vascular adhesion depends on shear rate. Scale of bar in experimental snapshots is 50 μm.

After calibrating and validating the numerical method for 220 nm particles (FIG. 18A), the method was employed to predict adhesion and retention of larger particles, e.g., particles with average diameters of 750 (FIG. 18B) and 1,000 nm. A similar procedure of injection and wash cycle was repeated for the larger particles, and concentration of particles at the bottom wall was measured before and after the washing stage. The initial concentration of nanoparticle solution during the injection stage was kept at 0.16 mg/mL for the larger particles. A summary of the various parameters used in the test studies is presented in Table 4.

TABLE 4
Test studies for chamber flow experiments and simulations

| | Polymer type* | Particle diameter^{†} (nm) | Flow conditions | Concentration^{‡} (mg/mL) |
|---|---|---|---|---|
| Small (~220 nm) | PLGA-b-HA | 206.1 ± 49.2 | 1. Injection at Re = 1 | 0.8 |
| | PLGA-b-HA-VHSPNKK | 233.1 ± 39.7 | 2. Wash at Re = 1 | |
| Medium (~750 nm) | PLGA-b-HA | 784.9 ± 23.2 | 3. Wash at Re = 5 | 0.16 |
| | PLGA-b-HA-VHSPNKK | 720.0 ± 18.5 | | |
| Large (1,000 nm) | PLGA-b-HA | 1,000 (simulated) | | 0.16 |
| | PLGA-b-HA-VHSPNKK | 1,000 (simulated) | | |

[symbol]

[symbol]

[symbol]

[symbol]

indicates data missing or illegible when filed

The simulations resulted in a nonuniform spatial distribution of the bound particles due to the stochastic nature of the adhesion model (FIG. 18). The pattern depended on the distribution of shear rate which affects the vascular deposition parameter (Eq. 16). Particle binding at the corners of the flow chamber was lower due to the low shear rates, which in turn resulted in smaller values of the vascular deposition parameter. Additionally, particles were effectively washed away near the inlet due to the inertial effect of the injected flow. The concentration of bound particles at the endothelium diminished at the end of wash stage because of dislodging hydrodynamic forces; however, there was no significant change in the spatial distribution of bound particles.

Effects of Particle Size and Chemistry on Particle Adhesion and Retention. To validate the simulation results, the transport of fluorescently labeled nanoparticles was measured: 220 nm and 750 nm diameter-PLGA-b-HA and PLGA-b-HA-VHSPNKK particles through a microfluidic chamber with a scale that was comparable to that of arteries (FIG. 19A). Endothelium inflamed by TNF-α was exposed to the media flow at Reynolds number of 1. According to confocal microscope images of endothelium exposed to the nanoparticle flow, unmodified PLGA-b-HA particles showed minimal adhesion to the endothelium, regardless of the particle diameter (FIG. 19B). In contrast, particles conjugated with VHSPNKK peptides exhibited increased adhesion to the inflamed endothelium, especially the particles with 750 nm diameter. In particular, PLGA-b-HA-VHSPNKK particles with 750 nm diameter displayed a substantial increase in adhesion compared to the particles with 220 nm diameter.

FIG. 19 depicts an experimental analysis of nanoparticle (NP) adhesion onto inflamed endothelium in a microfluidic chamber. (A) Schematic illustration of a microfluidic device in which endothelial cells were exposed to controlled flow. Endothelial cells were activated to up-regulate VCAM-1 expression. NPs with diameters of 220 (B) and 750 nm (C) were injected into the microfluidic chamber at Re of 1. NPs attached to endothelium were imaged (noted with “Injection”). Subsequently, NPs were exposed to the wash flow at Re of 1 and 5, and the NPs left on the endothelium were imaged again (noted with “Wash”). The endothelial cell nuclei were labeled with Hoechst 33342 (Blue), and the NPs were labeled with Alexa-488 (Green). (Scale bar, 50 μm).

The detachment of nanoparticles under external media flow at Re of 1 and 5 was also experimentally assessed. Nanoparticles adhered to the inflamed endothelium were first exposed to the external flow without suspended particles. Most PLGA-b-HA nanoparticles were washed out regardless of the size and Re (FIGS. 19A and B). In contrast, PLGA-b-HA-VHSPNKK nanoparticles displayed a dependency of the retention on the particle size and Re. Specifically, approximately 90% of particles with 750 nm diameter remained on the endothelium after exposure to the media flow with Re of 1. Increasing Re to 5 decreased the retention rate of particles significantly. The smaller particles with 220 nm diameter were detached at Re of both 1 and 5, despite the presence of VHSPNKK peptides.

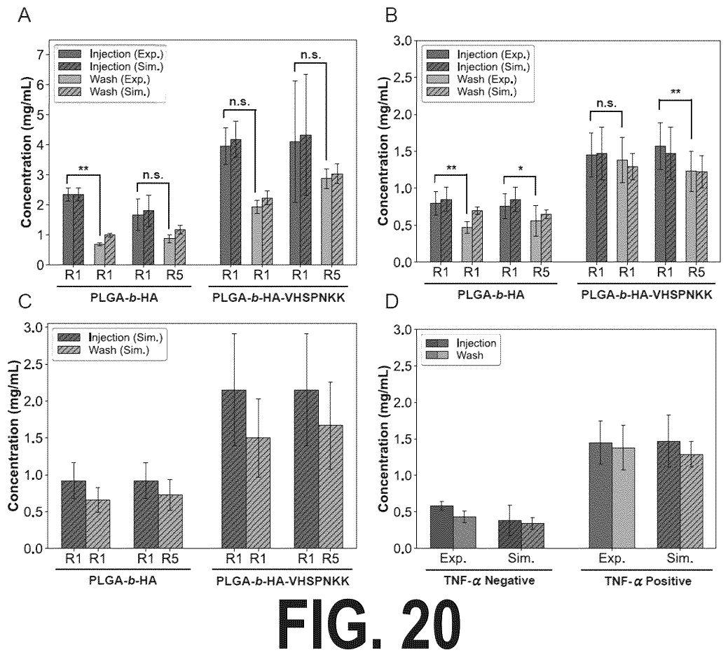

Whether the increased adhesion and retention of the larger PLGA-b-HA-VHSPNKK nanoparticles was specific to the inflamed endothelium was also assessed by comparing them with the same particles flowing through the microfluidic chamber where endothelium was not exposed to TNF-α (FIG. 20D). TNF-α-induced endothelium had 132% higher density of VCAM receptors, which resulted in three times higher adhesion and retention of particles. The particles demonstrated insignificant adhesion to the noninflamed endothelium due to the negligible expression of VCAM, as also confirmed by the simulations.

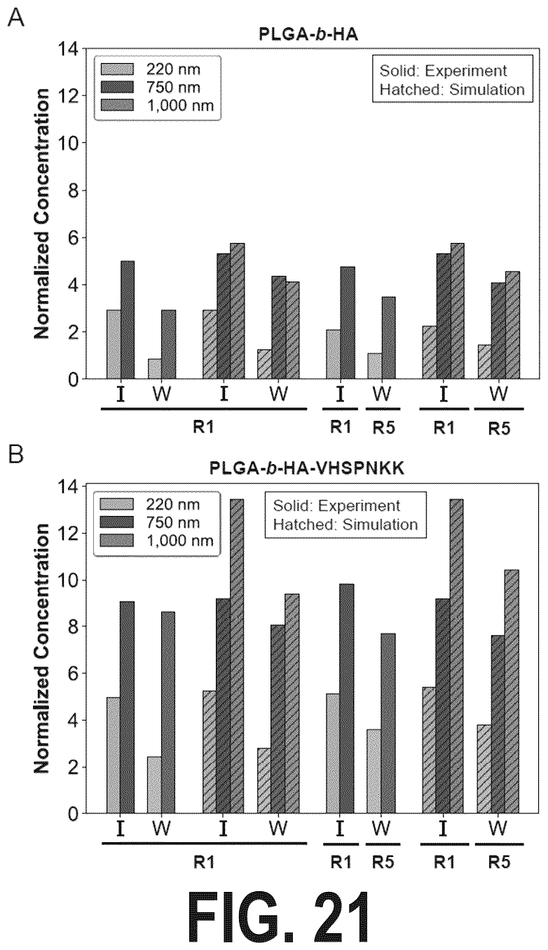

Comparative Analysis of Numerical Simulation and Experimentation. Experiments were performed using 220 nm particles, with the results used to calibrate the computational model (e.g., by setting the various parameters thereof according to experimentally measured values). The concentration of nanoparticles on the endothelium was determined by running an image analysis of confocal images displayed in FIG. 19. With 220 nm diameter particles, both numerical simulations and experimental measurements showed that VHSPNKK peptide conjugation to the PLGA-b-HA particles made an approximately two-fold increase in the number of particles bound to the inflamed endothelium (FIGS. 20A and 6). In addition, both experiments and simulations revealed that external flow, particularly at Re of 1, separated PLGA-b-HA nanoparticles from the inflamed endothelium. Increasing the Re from 1 to 5 increased the particle retention percentage, which is a mass ratio between nanoparticles remaining on the endothelium after external flow and those on the endothelium before the external flow, from 50% to 77%. In contrast, the 750 nm diameter PLGAb-HA-VHSPNKK particles exhibited insignificant loss following the external flow at Re of 1, which was also predicted by the numerical analysis (FIGS. 20B and 21). When the flow rate increased to Re=5, the retention of 750 nm PLGA-b-HA-VHSPNKK particles dropped slightly, but still as significantly as 79%. According to the numerical simulations, a further increase in particle diameter to 1,000 nm would not significantly change the adhesion and retention of particles (FIG. 20C).

FIG. 20 depicts a comparative analysis of the adhesion and retention of nanoparticles between numerical simulation and experimentation. The concentration of nanoparticles at the center of the blood vessel-simulating channel bottom was quantified at the end of nanoparticle injection (for adhesion) and after the wash flow with particle-free media (for retention). (A) Concentration of PLGA-b-HA and PLGA-b-HA-VHSPNKK particles with an average diameter of 220 nm. The numerical method was calibrated with the experiments conducted at Re=1. (B) Concentration for PLGA-b-HA and PLGA-b-HA-VHSPNKK particles with an average diameter of 750 nm. (C) Numerically predicted concentration values for PLGA-b-HA and PLGA-b-HA-VHSPNKK particles with an average diameter of 1,000 nm. (D) Effect of TNF-α treatment of endothelial cells on adhesion and retention of 750 nm-diameter PLGA-b-HA-VHSPNKK particles. The adhesion and retention were tested with media flow at Re of 1. In (A-C), R1 and R5 on the x-axis represent Reynolds numbers 1 and 5 of the media flow, respectively. The error bars represent a SE in each dataset. n.s., *, and ** indicate not statistically significant with P>0.05, P<0.05, and P<0.005, respectively.

Subsequently, using the calibrated model (as above), the behavior of 750 nm particles was simulated, and then the accuracy of the model described herein was evaluated via in vitro experimentation. The model predicted that the fluid flow resulted in higher loss of same-sized PLGA-b-HA particles due to dislodging hydrodynamic forces. The numerical model and experiments also predicted that the VHSPNKK peptides conjugated to the PLGA-b-HA particles would act to increase particle adhesion to the target inflamed endothelium but have a smaller influence on the particle retention (FIGS. 20 and 21). Since the model simulates the process at a continuum scale, individual particles and their clusters at the nanoscale were not explicitly generated. However, the macro distribution of nanoparticle concentration over the endothelium exhibited a stochastic pattern that was similar to the experimental snapshots (FIG. 18), and the concentration values averaged over a small patch at the center of the channel are in agreement with the experiments (FIG. 20).

By validating the model with experimentation, it was found that PLGA-b-HA-VHSPNKK nanoparticles with 750 nm diameter remained on the target endothelium better than the smaller particles in the face of the flow generated shear force, as evident from their higher retention percentages. This result, also observed in the vitro experimentation, may be attributable to the increased surface area of the larger particles, which increases the number of VHSPNKK-VCAM1 bonds and, in turn, the adhesion strength of the particles. Note that PLGA-b-HA nanoparticles free of VHSPNKK peptides did not show an insignificant dependency of particle retention on the particle size. The model also accurately predicted that PLGA-b-HA-VHSPNKK peptides would show increased adhesion and insignificant detachment on the endothelium that was treated with TNF-α. This was also observed in the in vitro experimentation, possibly related to the overexpression of VCAM-1 receptors. The larger size of nanoparticles also provides synergistic increase in the particle retention level along with the strong interaction between larger PLGA-b-HA-VHSPNKK nanoparticles and inflamed endothelium. This synergy was further amplified by the normal force exerted by the flow. Despite the larger shear forces experienced at higher Reynolds numbers, the increased size of PLGA-b-HA-VHSPNKK nanoparticles allowed them to resist detachment and maintain adhesion to the inflamed endothelium. The combination of strong binding interactions between the nanoparticles and the inflamed endothelium, along with the ability to withstand higher shear forces due to their larger size, facilitated the adhesion and prolonged tethering of particles to the inflamed sites.

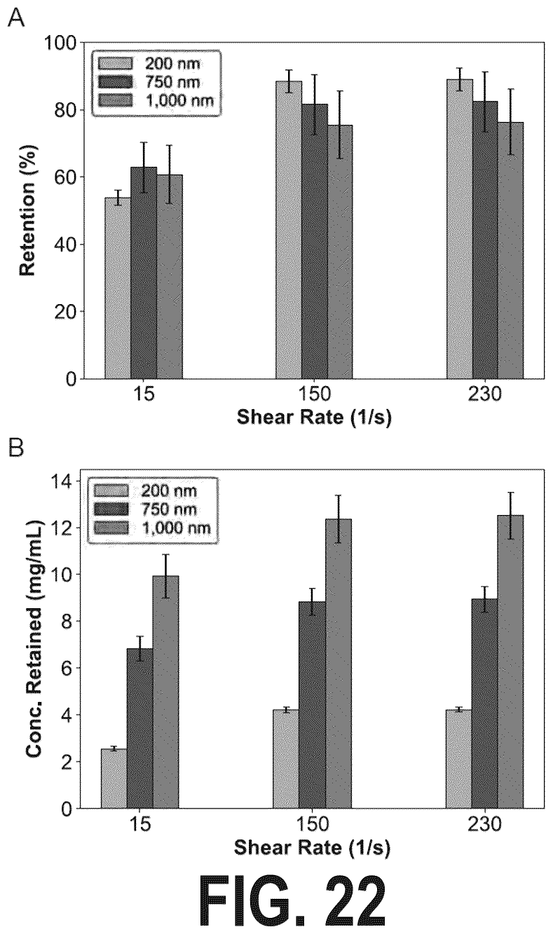

To mimic higher physiological shear rates, a microchannel of dimensions 0.1×0.1×5 mm was simulated with shear rates of 15, 150, and 230 s⁻¹. All three particle sizes (220, 750, and 1,000 nm) were studied across injection and wash stages. During the injection stage, 1 mL solution of PLGA-b-HA-VHSPNKK particles with a concentration of 1 mg/mL was injected into the chamber. Subsequently, a wash stage was simulated in which 3 mL of particle-free media was flown through the channel to study the detachment of bound particles under an external flow. The higher adhesion force in the larger-sized particles resulted in higher concentration at the targeted site as compared to the smaller particles. As a result, the remaining concentration after the wash cycle was higher when compared to the smaller size particles (FIG. 22B), even though the percentage retention of larger particles after adhesion decreased at higher shear rates (150, 230 s⁻¹) due to greater dislodging forces (FIG. 22A). This agrees with the earlier results (FIG. 21) that larger particles performed better in adhesion and retention at the endothelium for particle sizes suitable for drug delivery applications and within physiologically relevant shear rates. The greater adhesion and retention of larger-sized particles may be explained by the biphasic behavior of the vascular adhesion parameter in Eq. 16. At lower shear rates, adhesion forces dominate, and the adhesion parameter increases exponentially as a function of shear rate. After an inflection point, dislodging forces take over and the opposite behavior is observed. For larger particles, the adhesion dominated range of shear rates decreases in size. However, within the range of relevant shear rates, even though the dislodging forces dominate at higher shear rates for 1,000 nm particles resulting in decreased adhesion, the value is still greater than the adhesion for lower-sized particles (220, 750 nm). This results in greater adhesion and retention of 1,000 nm particles across all shear rates tested, as shown in FIG. 22B.

FIG. 21 depicts a normalized analysis of the particle size effect on adhesion and retention of (A) PLGA-b-HA particles and (B) PLGA-b-HA-VHSPNKK particles. Particle concentration was normalized w.r.t. the concentration of the injected nanoparticle solution during the injection stage. R1 and R5 represent Reynolds numbers 1 and 5 of the media flow, respectively (I=Injection flow with particle-laden media; W=Wash flow with particle-free media).

FIG. 22 depicts the effect of size on retention of injected particles across physiologically relevant shear rates. (A) Percentage retention of the attached particle (during the injection stage) after wash flow. The difference in retention rates across different particle sizes was not statistically significant for any particular shear rate. (B) Concentration of retained particles at the end of wash stage. Concentration values of different particle sizes differed statistically significantly at all the shear rates.

TABLE 5
Parameters for the particle-cell adhesion model

| Parameter | | Value | | Reference |
|---|---|---|---|---|
| 1. | Mean surface density of receptors, mᵣ | 10¹³ | #/m² | (25) |
| 2. | Mean surface density of ligands, mₗ | 10¹⁵ | #/m² | (25) |
| 3. | Ligand-receptor affinity constant at | 0.069 | μm² | (34) |
| | zero load, Kₐ⁰ | | | |
| 4. | Distance between particles and | 45 | nm | — |
| | endothelial cell layer at equilibrium, Δ | | | |
| 5. | Ligand-receptor bond length, λ | 5 | nm | — |
| 6. | Drag coefficient for spherical | 1.668 | | — |
| | particles, F^{s} | | | |

### III. EXAMPLE METHODS

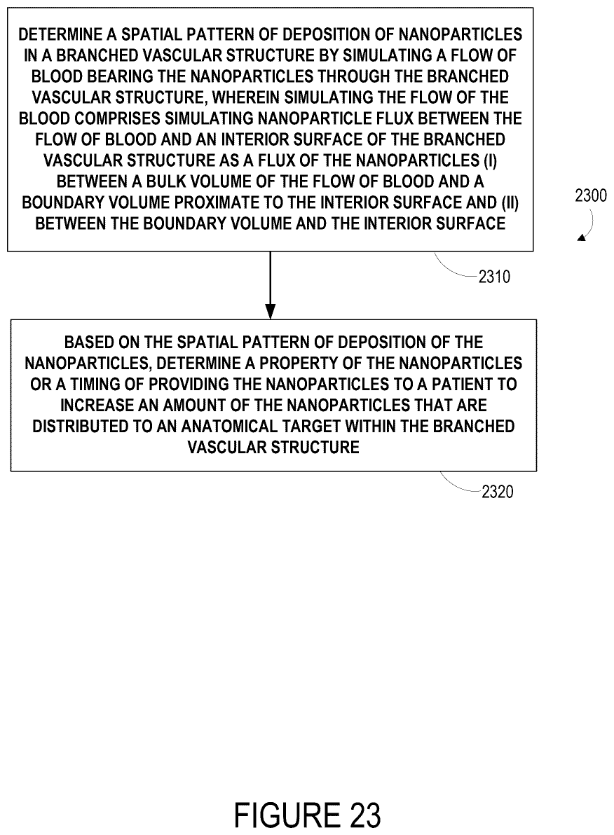

FIG. 23 is a flowchart of an example method 2300 for accurately simulating the deposition/washing of nanoparticles in human or animal vasculature, e.g., in order to tailor nanoparticle configuration or dosing patterns to improve deposition at targeted tissue(s). The method 2300 includes determining a spatial pattern of deposition of nanoparticles in a branched vascular structure by simulating a flow of blood bearing the nanoparticles through the branched vascular structure, wherein simulating the flow of the blood comprises simulating nanoparticle flux between the flow of blood and an interior surface of the branched vascular structure as a flux of the nanoparticles (i) between a bulk volume of the flow of blood and a boundary volume proximate to the interior surface and (ii) between the boundary volume and the interior surface (2310). The method 2300 additionally includes based on the spatial pattern of deposition of the nanoparticles, determining a property of the nanoparticles or a timing of providing the nanoparticles to a patient to increase an amount of the nanoparticles that are distributed to an anatomical target within the branched vascular structure (2320). The method 2300 could include additional or alternative features.

### IV. CONCLUSION

It should be understood that arrangements described herein are for purposes of example only. As such, those skilled in the art will appreciate that other arrangements and other elements (e.g., machines, interfaces, operations, orders, and groupings of operations, etc.) can be used instead, and some elements may be omitted altogether according to the desired results. Further, many of the elements that are described are functional entities that may be implemented as discrete or distributed components or in conjunction with other components, in any suitable combination and location, or other structural elements described as independent structures may be combined.

While various aspects and implementations have been disclosed herein, other aspects and implementations will be apparent to those skilled in the art. The various aspects and implementations disclosed herein are for purposes of illustration and are not intended to be limiting, with the true scope being indicated by the following claims, along with the full scope of equivalents to which such claims are entitled. It is also to be understood that the terminology used herein is for the purpose of describing particular implementations only, and is not intended to be limiting.

We claim:

1. A method comprising:
determining a spatial pattern of deposition of nanoparticles in a branched vascular structure by simulating a flow of blood bearing the nanoparticles through the branched vascular structure, wherein simulating the flow of the blood comprises simulating nanoparticle flux between the flow of blood and an interior surface of the branched vascular structure as a flux of the nanoparticles (i) between a bulk volume of the flow of blood and a boundary volume proximate to the interior surface and (ii) between the boundary volume and the interior surface; and
based on the spatial pattern of deposition of the nanoparticles, determining a property of the nanoparticles or a timing of providing the nanoparticles to a patient to increase an amount of the nanoparticles that are distributed to an anatomical target within the branched vascular structure.

2. The method of claim 1, further comprising:
based on the determined property of the nanoparticles or timing, providing nanoparticles having the determined property to a patient or providing nanoparticles to the patient at the determined timing.

3. The method of claim 2, wherein the patient is experiencing a stroke, wherein the branched vascular structure has a structure of narrowed or missing branches that simulates a stroke, and wherein the anatomical target comprises a plurality of locations of the branched vascular structure proximate to bifurcations of the branched vascular structure at the base of the narrowed or missing branches.

4. The method of claim 3, wherein the branched vascular structure corresponds to a structure of vasculature of the patient's brain.

5. The method of claim 3, wherein the branched vascular structure corresponds to a structure of vasculature of a representative person's brain.

6. The method of claim 2, wherein the patient has a tumor, wherein the branched vascular structure corresponds to a structure of vasculature of the patient's body, and wherein the anatomical target comprises a location of the tumor relative to the branched vascular structure.

7. The method of claim 6, wherein simulating the flow of the blood comprises simulating the flow of blood in a manner that simulates a circadian pattern of changes in the flow of blood, and wherein determining a timing of providing the nanoparticles to the patient based on the spatial pattern of deposition of the nanoparticles comprises determining a timing of providing the nanoparticles relative to a phase of the circadian pattern in the patient.

8. The method of claim 6, wherein simulating the flow of the blood comprises simulating the flow of blood for a plurality of different binding targets of the nanoparticles.

9. The method of claim 6, wherein simulating the flow of the blood comprises simulating the flow of blood for a plurality of different sizes of the nanoparticles.

10. The method of claim 1, wherein simulating nanoparticle flux between the flow of blood and the interior surface of the branched vascular structure as a flux of the nanoparticles comprises simulating the flux of the nanoparticles in a manner that is dependent on local concentration of the nanoparticles and the flow of blood at the interior surface normal to the interior surface.

11. The method of claim 10, wherein simulating nanoparticle flux between the flow of blood and the interior surface of the branched vascular structure as a flux of the nanoparticles comprises simulating flux of the nanoparticles between the boundary volume and the interior surface in a manner that is dependent upon shear within the blood near the interior surface.

12. The method of claim 11, wherein simulating flux of the nanoparticles between the boundary volume and the interior surface comprises simulating surface roughness of the interior using a friction term that is only activated within a specified distance of the interior surface.

13. The method of claim 10, herein simulating nanoparticle flux between the flow of blood and the interior surface of the branched vascular structure as a flux of the nanoparticles comprises simulating mixing of nanoparticles near the interior surface in a manner that is dependent upon shear within the blood near the interior surface.

14. The method of claim 1, wherein simulating the flow of the blood comprises simulating the resistances of the ends of the branched vascular structure as static fluid resistances.

15. The method of claim 1, further comprising:
obtaining a scan of a structure of vasculature of a patient's body, wherein the branched vascular structure corresponds to the structure of vasculature of the patient's body.

16. The method of claim 15, wherein determining a property of the nanoparticles based on the spatial pattern of deposition of the nanoparticles comprises, based on the spatial pattern of deposition of the nanoparticles, selecting a nanoparticle for the patient from a set of nanoparticles that differ with respect to size or binding target.

17. The method of claim 15, further comprising:
obtaining a measurement of a circadian pattern of changes in the flow of blood of the patient, wherein simulating the flow of the blood comprises simulating the flow of blood in a manner that simulates the circadian pattern of changes in the flow of blood of the patient.

18. The method of claim 1, wherein simulating the flow of the blood comprises simulating the flow of blood in a manner that simulates variation over time of a pressure and flow rate of the blood into the branched vascular structure.

19. The method of claim 18, further comprising:
obtaining, for a patient, a time-varying blood pressure profile, wherein simulating the flow of blood in a manner that simulates variation over time the pressure and flow rate of the blood into the branched vascular structure comprises simulating the flow of blood in a manner that corresponds to the time-varying blood pressure profile of the patient.

20. A non-transitory computer readable medium having stored thereon program instructions executable by at least one processor to cause the at least one processor to perform operations comprising:
determining a spatial pattern of deposition of nanoparticles in a branched vascular structure by simulating a flow of blood bearing the nanoparticles through the branched vascular structure, wherein simulating the flow of the blood comprises simulating nanoparticle flux between the flow of blood and an interior surface of the branched vascular structure as a flux of the nanoparticles (i) between a bulk volume of the flow of blood and a boundary volume proximate to the interior surface and (ii) between the boundary volume and the interior surface; and
based on the spatial pattern of deposition of the nanoparticles, determining a property of the nanoparticles or a timing of providing the nanoparticles to a patient to increase an amount of the nanoparticles that are distributed to an anatomical target within the branched vascular structure.
